(19) United States
(12) Patent Application Publication
(10) Pub. No.: US 2024/0398759 A1
(43) Pub. Date: Dec. 5, 2024

(54) INHIBITING SEROTONIN TRANSPORTER PROTEIN (5-HTT)

(71) Applicant: Sensorium Therapeutics, Inc., Boston, MA (US)

(72) Inventors: Michael S. Placzek, Tewksbury, MA (US); Jacob M. Hooker, Winchester, MA (US)

(21) Appl. No.: 18/807,015

(22) Filed: Aug. 16, 2024

Related U.S. Application Data
(63) Continuation of application No. PCT/US23/85278, filed on Dec. 21, 2023.
(60) Provisional application No. 63/434,726, filed on Dec. 22, 2022.

(51) Int. Cl. A61K 31/404; A61P 25/24

(52) U.S. Cl. CPC A61K 31/404; A61P 25/24

(57) ABSTRACT

Disclosed are compositions comprising mesembranol, such as (−) mesembranol or (−) 6-epi-mesembranol. The disclosed compositions can be used in methods of inhibiting SERT. Methods of treatment for anxiety or depression are also provided, including methods comprising the oral administration to a subject in need thereof a therapeutically effective amount of certain pharmaceutical compositions.

This application claims the benefit of priority to U.S. Provisional Patent Application Ser. No. 63/434,726, filed Dec. 22, 2022; the contents of which are hereby incorporated by reference in their entirety.

### TECHNICAL FIELD

The present disclosure relates to the field of medicine, including the discovery of alkaloid compounds useful for inhibiting the serotonin transporter protein (5-HTT).

### BACKGROUND

Plants of the genus Sceletium contain indole alkaloids having biological activity useful in treating mental health conditions such as mild to moderate depression. Natural extracts of Sceletium tortuosum, an indigenous herb of South Africa also referred to as “kougoed”, “channa” or “kanna,” can contain the pharmacologically active alkaloids. Mesembrine, mesembrenone and mesembrenol, among the alkaloids shown below, are present in Sceletium tortuosum extracts used for treatment of anxiety, stress and mental health conditions.

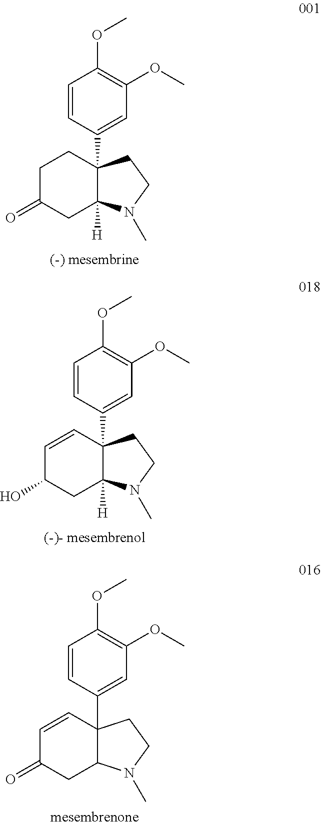 [SMILES: COc1ccc([C@@]23CCC(=O)C[C@@H]2N(C)CC3)cc1OC ; COc1ccc([C@@]23C=C[C@@H](O)C[C@@H]2N(C)CC3)cc1OC ; COc1ccc(C23C=CC(=O)CC2N(C)CC3)cc1OC]

Mesembranol is not well-characterized pharmacologically and concentration of this alkaloid in plant extracts are comparatively low relative to the other biologically active Sceletium tortuosum alkaloids. Mesembranol has little to no abundance in extracts derived from Sceletium tortuosum plant. FIGS. 1A-1C represent liquid chromatograpy mass spectrometry (LC-MS) chemical fingerprints of alkaloids present in sceletium tortuosum plants taken from 3 different regions within South Africa, where sceletium tortuosum is native (J. Zhao et al., Phytochemistry, 2018). In each chromatogram, the peak at 3.77 minutes is attributed to mesembranol. Samples from Northern Cape (FIG. 1A) and Western Cape 1 (FIG. 1B) demonstrated only trace amounts of mesembranol present. Mesembranol was not the major alkaloid present and has only modest abundance in Western Cape 2 samples (4^{th} most abundant alkaloid in Western Cape 2 samples; FIG. 1C). These data indicate that Sceletium tortuosum plant extraction is unlikely to yield sufficient quantities of mesembranol to provide therapeutic benefit.

In addition, extracts derived from Sceletium tortuosum contain a number of biologically active alkaloids with a range of pharmacological effects. For example, reported pharmacological activity of Sceletium tortuosum extracts includes serotonin transporter inhibition (5-HTT), phosphodiesterase-4 inhibition (PDE4), monoamine oxidase A (MAO-A) inhibitory activity, acetylcholine esterase (AChE) inhibitory activity, GABA receptor binding activity, opioid receptor binding activity, dopamine transporter inhibtion, AMPA receptor modulation, effects on vesicular monoamine transporter-2 (VMAT-2) and subsequent monoamine release.

An analysis of a standardized commercial extract of Sceletium tortuosum was reported in 2011 (obtained as a product under the tradename, Zembrin®) as having 0.35%-0.45% total alkaloids, with mesembrenone and mesembrenol comprising ≥60%, and mesembrine contributing <20% (See Harvey et al., “Pharmacological actions of the South African medicinal and functional food plant Sceletium tortuosum and its principal alkaloids,” Journal of Ethnopharmacology 137 (2011) 1124-1129 and Murbach et. al., “A toxicological safety assessment of a standardized extract of Sceletium tortuosum (Zembrin®) in rats, “Food and Chemical Toxicology 74 (2014) 190-199. The extract gave >80% inhibition at serotonin (5-HT) transporter with potency of the isolated alkaloids at the 5-HT transporter reported as shown in Table A below (Harvey et al., 2011). Referring to the data in Table A, concentration-dependent inhibition was found, with mesembrine being the more active compound (i.e., 20 times more potent than mesembrenone and 87 times more active than mesembrenol) in the 5-HT transporter assay. A toxicological safety assessment of this standardized extract was subsequently reported in 2014 (Murbach et al., 2014).

TABLE A
Summary of analysis of the concentration response curves of
alkaloids on binding to the 5-HT transporter (Harvey et al., 2011)

| | 5-HT transporter (SERT) | |
|---|---|---|
| Compound | Ki (nM) | nH |
| Mesembrine | 1.4 | 1.0 |
| Mesembrenone | 27 | 1.0 |
| Mesembrenol | 62 | 1.1 |

However, bioactive plant extracts for therapeutic consumption can vary widely both seasonally and between different Sceletium tortuosum plants, and fail to provide a sufficiently reproducible and stable phytochemical profile of desired biologically active components. Plants of the genus Sceletium and extracts thereof can vary widely in terms of the total alkaloid content, as well as the chemistry and relative concentrations of individual Sceletium plant derived alkaloids. In addition, it has been reported that mesembranol concentrations in sceletium tortuosum can vary across regions of South Africa, and mesembranol abundance was relatively low in most plant extracts that were tested. Lastly, sceletium alkaloids may be unstable under a variety of conditions that can occur during extraction from plant material, as well as during storage and formulation of the extract.

In Sceletium tortuosum extract, mesembranol, and its isomer 6-epi-mesembranol, have low concentrations compared to the other major alkaloids. Therefore, mesembranol and 6-epi-mesembranol have not been well characterized in the scientific literature. The pharmacological activity and selectivity of mesembranol or 6-epi-mesembranol have not been reported. Their therapeutic use has been limited by the low abundance, variability and instability of these alkaloids in natural extract products, and the instability and pharmacokinetic profile of these compounds as obtained from natural products. Naturally occurring mesembranol and 6-epi-mesembranol are serotonin transporter inhibitors with high specificity relative to the other aforementioned pharmacological targets. Sceletium tortuosum alkaloid extract does not yield sufficient levels of mesembranol and 6-epi-mesembranol relative to other components, and the extract could also exert other additional pharmacological effects that are undesirable for a therapeutic regimen for anxiety or depression.

There remains an unmet need for pharmaceutical compositions comprising higher purity, predictable, stable and reproducible forms of therapeutic alkaloid compounds such as mesembranol and 6-epi-mesembranol. In addition, there is a need for oral pharmaceutical compositions providing pure therapeutic alkaloid compositions having desired pharmacokinetic properties upon administration. Finally, there is an unmet need for pharmaceutical compositions comprising markedly different properties than the naturally occurring compositions obtained from plant extracts.

### SUMMARY

The Applicants have discovered new and useful methods of using mesembranol (i.e., (−) mesembranol or (−) 6-epi-mesembranol). In some embodiments, mesembranol (Compound 18) or (−) 6-epi-mesembranol (Compound 19) can be used to selectively inhibit the serotonin transporter (5-HTT) while avoiding inhibition of phosphodiesterase-4 (PDE4) at or near levels found in other pharmacologically active alkaloids identified in Sceletium tortuosum extracts.

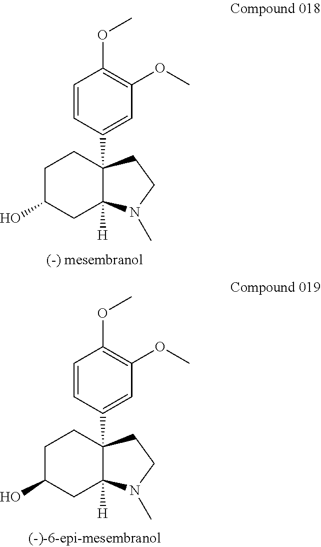 [SMILES: COc1ccc([C@@]23CC[C@@H](O)C[C@@H]2N(C)CC3)cc1OC ; COc1ccc([C@@]23CC[C@H](O)C[C@@H]2N(C)CC3)cc1OC]

In some embodiments, methods of using (−) mesembranol or (−) 6-epi-mesembranol are based in part on the discovery (−) mesembranol or (−) 6-epi-mesembranol may be used to provide a mechanism of therapeutic action that is similar to a selective serotonin reuptake inhibitor (SSRI).

In some embodiments, methods of using (−) 6-epi-mesembranol are based in part on the discovery of different pharmacokinetic data for (−) 6-epi-mesembranol (Compound 019) and (−) mesembranol (Compound 018), including a half-life that is about three times greater for Compound 019 compared to Compound 018 and about twice as long as the half-life measured for mesembrine (Compound 001) (as measured after intravenous administration to dogs according to Example 7).

In some embodiments, (−) 6-epi-mesembranol (Compound 19) can be used to selectively inhibit the serotonin transporter (5-HTT) while avoiding inhibition of phosphodiesterase-4 (PDE4) at or near levels found in other pharmacologically active alkaloids identified in Sceletium tortuosum extracts. In some embodiments, (−) 6-epi-mesembranol (Compound 19) can be used for the treatment of anxiety or depression. In certain embodiments, a method of treating anxiety and depression, comprises orally administering to a subject in need thereof a therapeutically effective amount of a pharmaceutical composition comprising Compound 019,

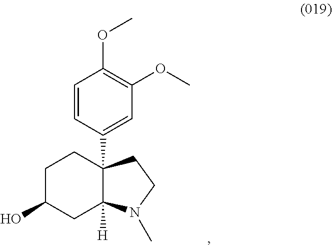 [SMILES: COc1ccc([C@@]23CC[C@H](O)C[C@@H]2N(C)CC3)cc1OC]

or a pharmaceutically acceptable salt thereof. In certain embodiments, the method is a method of treating Major Depressive Disorder (MDD). In certain embodiments, the method is a method is a method of treating General Anxiety Disorder (GAD).

In certain embodiments, Compound 019, or the pharmaceutically acceptable salt thereof, is administered as a capsule or a tablet. In certain embodiments, Compound 019, or the pharmaceutically acceptable salt thereof, is administered once or twice per day. In certain embodiments, the pharmaceutical composition contains no more than about 1% of an alkaloid selected from (−) mesembrine, mesembrenone, mesembrenol, or a combination thereof.

In certain embodiments, a method of inhibiting SERT, comprises administering to a mammal in need thereof a therapeutically effective amount of

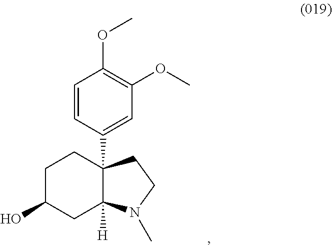 [SMILES: COc1ccc([C@@]23CC[C@H](O)C[C@@H]2N(C)CC3)cc1OC]

or a pharmaceutically acceptable salt thereof.

In certain embodiments, a pharmaceutical composition, comprises

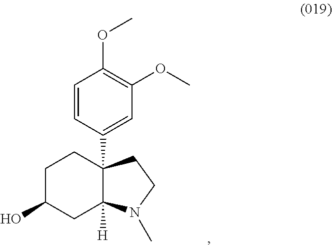 [SMILES: COc1ccc([C@@]23CC[C@H](O)C[C@@H]2N(C)CC3)cc1OC]

or a pharmaceutically acceptable salt thereof, wherein the pharmaceutical composition contains no more than about 3% of an alkaloid selected from (−) mesembrine, mesembrenone, mesembrenol, or a combination thereof; and Compound 019 is at least 90% of the total alkaloid content in the pharmaceutical composition. In certain embodiments, the ratio of PDE4/SERT inhibition values of the pharmaceutical composition is at least 10:1. In certain embodiments, the Compound 019 has a half-life of at least 30 minutes in a human hepatocyte assay of the pharmaceutical composition. In certain embodiments, the pharmaceutical composition has an IC₅₀ for SERT of less than about 30 nM. In certain embodiments, the pharmaceutical composition has a % inhibition of less than about 6% for at least one of PDE4A1A, PDE4B2, PDE4C1, and PDE4D2. In certain embodiments, the ratio of PDE4/SERT inhibition values of the pharmaceutical composition is at least 10:1.

In certain embodiments, a method of inhibiting SERT without inhibiting PDE4, comprising administering to mammal in need thereof a therapeutically effective amount of a pharmaceutical composition comprising Compound 019 or a pharmaceutically acceptable salt thereof. In certain embodiments, a composition comprising Compound 019, or a pharmaceutically acceptable salt thereof, and less than about 0.5% of mesembrine or mesembrenone as measured by HPLC.

A method of treating anxiety or depression, can comprise orally administering to a subject in need thereof a therapeutically effective amount of a pharmaceutical composition comprising Compound 019,

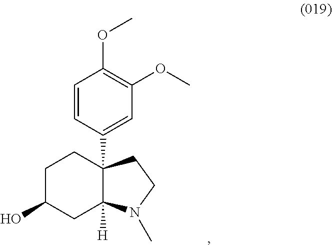 [SMILES: COc1ccc([C@@]23CC[C@H](O)C[C@@H]2N(C)CC3)cc1OC]

or a pharmaceutically acceptable salt thereof. In some embodiments, the method is a method of treating Major Depressive Disorder (MDD), or a method of treating General Anxiety Disorder (GAD).

In some embodiments, Compound 019, or the pharmaceutically acceptable salt thereof, is administered as a capsule or a tablet. In some embodiments, Compound 019, or the pharmaceutically acceptable salt thereof, is administered once or twice per day. In some embodiments, the pharmaceutical composition contains no more than about 1% of an alkaloid selected from (−) mesembrine, mesembrenone, mesembrenol, or a combination thereof.

In some embodiments, a method of inhibiting SERT, comprising administering to a mammal in need thereof a therapeutically effective amount of

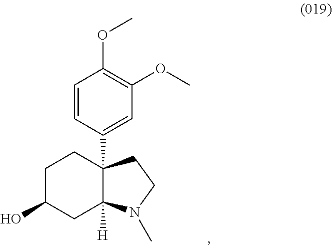 [SMILES: COc1ccc([C@@]23CC[C@H](O)C[C@@H]2N(C)CC3)cc1OC]

or a pharmaceutically acceptable salt thereof.

In some embodiments, a pharmaceutical composition, is provided comprising

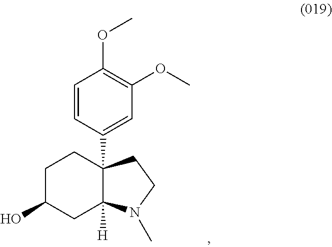 [SMILES: COc1ccc([C@@]23CC[C@H](O)C[C@@H]2N(C)CC3)cc1OC]

or a pharmaceutically acceptable salt thereof, wherein the pharmaceutical composition contains no more than about 3% of an alkaloid selected from (−) mesembrine, mesembrenone, mesembrenol, or a combination thereof; and. Compound 019 is at least 90% of the total alkaloid content in the pharmaceutical composition.

In some embodiments, the ratio of PDE4/SERT inhibition values of the pharmaceutical composition is at least 10:1. In some embodiments, the Compound 019 has a half-life of at least 30 minutes in a human hepatocyte assay of the pharmaceutical composition. In some embodiments, the pharmaceutical composition has an IC₅₀ for SERT of less than about 30 nM. In some embodiments, the pharmaceutical composition has a % inhibition of less than about 6% for at least one of PDE4A1A, PDE4B2, PDE4C1, and PDE4D2. In some embodiments, the ratio of PDE4/SERT inhibition values of the pharmaceutical composition is at least 10:1.

In some embodiments, a method of inhibiting SERT without inhibiting PDE4, comprises administering to mammal in need thereof a therapeutically effective amount of a pharmaceutical composition comprising Compound 019 or a pharmaceutically acceptable salt thereof.

In some embodiments, a composition is provided comprising Compound 019, or a pharmaceutically acceptable salt thereof, wherein the composition comprises less than about 0.5% of mesembrine or mesembrenone as measured by HPLC.

In some embodiments, a method of treating anxiety or depression, comprises orally administering to a subject in need thereof a therapeutically effective amount of a pharmaceutical composition comprising Compound 018,

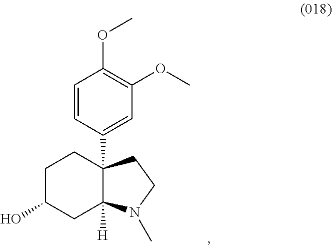 [SMILES: COc1ccc([C@@]23CC[C@@H](O)C[C@@H]2N(C)CC3)cc1OC]

or a pharmaceutically acceptable salt thereof.

Numerous other embodiments are further provided that can be applied to any aspect of the present invention described herein.

### BRIEF DESCRIPTIONS OF THE DRAWINGS

FIG. 1A is a chromatogram of the Northern Cape sample, as reproduced from J. Zhao et al., Phytochemistry, 2018.

FIG. 1B is a chromatogram of the Western Cape 1 sample, as reproduced from J. Zhao et al., Phytochemistry, 2018.

FIG. 1C is a chromatogram of the Western Cape 2 sample, as reproduced from J. Zhao et al., Phytochemistry, 2018.

FIG. 2A is a graph showing the compound stability of (−) mesembrine (triangles) compared to (−) mesembranol (squares) in human hepatocytes.

FIG. 2B is a graph showing the compound stability of (−) mesembrine (triangles) compared to (−) 6-epi-mesembranol (circles) in human hepatocytes.

FIG. 3 is a graph showing the pharnmacokineticc profile of Compound 018 and Compound 019 evaluated in dogs, showing the plasma concentrations (nM) of Compound 018, Compound 019 and Compound 001 following IV administration of compound at 2 mg/kg.

FIG. 4 is a graph showing the plasma concentrations (nM) of Compound 018, Compound 019, and Compound 001 following PO administration of compounds at 10 mg/kg.

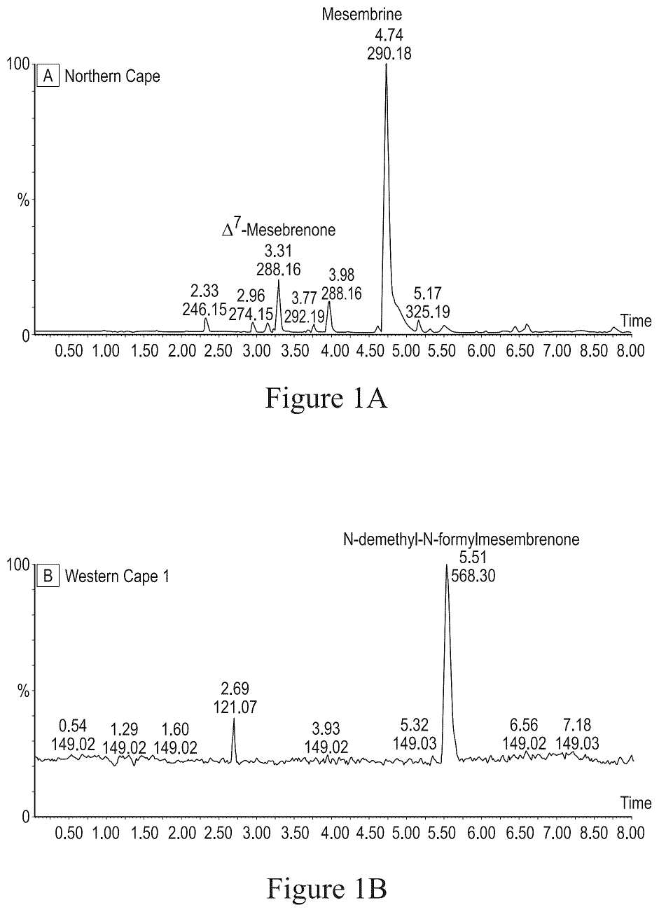

### DETAILED DESCRIPTION

Described herein are compositions comprising a compound selected from mesembranol or 6-epi mesembranol, or a pharmaceutically acceptable salt thereof, and methods of using (−) mesembranol and (−) 6-epi-mesembranol.

The Applicants have discovered new and useful methods of using mesembranol (i.e., (−) mesembranol or (−) 6-epi-mesembranol). In some embodiments, mesembranol (Compound 18) or (−) 6-epi-mesembranol (Compound 19) can be used to selectively inhibit the serotonin transporter (5-HTT) while avoiding inhibition of phosphodiesterase-4 (PDE4) at or near levels found in other pharmacologically active alkaloids identified in Sceletium tortuosum extracts.

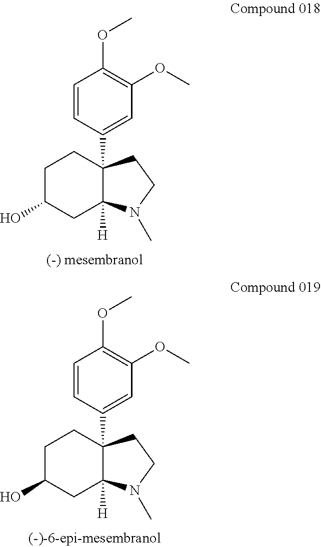 [SMILES: COc1ccc([C@@]23CC[C@@H](O)C[C@@H]2N(C)CC3)cc1OC ; COc1ccc([C@@]23CC[C@H](O)C[C@@H]2N(C)CC3)cc1OC]

In some embodiments, methods of using (−) mesembranol or (−) 6-epi-mesembranol are based in part on the discovery (−) mesembranol or (−) 6-epi-mesembranol may be used to provide a mechanism of therapeutic action that is similar to a selective serotonin reuptake inhibitor (SSRI).

In some embodiments, a method of treating a mental health disorder, comprising administering to a mammal in need thereof an effective amount of a compound selected from (−) mesembranol and (−) 6-epi-mesembranol, or a salt thereof. In certain embodiments, the compound is (−) mesembranol. In certain embodiments, the compound is (−) 6-epi-mesembranol.

In certain embodiments, the present disclosure provides a method of inhibiting SERT, comprising administering to a mammal in need thereof a therapeutically of a compound selected from (−) mesembranol and (−) 6-epi-mesembranol, or a salt thereof. In certain embodiments, the mesembranol is (−) mesembranol. In certain embodiments, the compound is (−) mesembranol. In certain embodiments, the compound is (−) 6-epi-mesembranol.

In certain embodiments, the present disclosure provides a method of inhibiting SERT, comprising administering to a mammal in need thereof a therapeutically effective amount of a pharmaceutical composition comprising of a compound selected from (−) mesembranol and (−) 6-epi-mesembranol, or a salt thereof. In certain embodiments, the compound is (−) mesembranol. In certain embodiments, the compound is (−) 6-epi-mesembranol.

In certain embodiments, a pharmaceutical composition comprises (−) 6-epi-mesembranol and a pharmaceutically acceptable excipient. In certain embodiments, a pharmaceutical composition comprises mesembranol and a pharmaceutically acceptable excipient. In certain embodiments, the present disclosure provides a method of treating a mental disorder, comprising administering to the subject a compound of the present disclosure. In certain embodiment, a method of inhibiting SERT without inhibiting PDE4, comprises administering to mammal in need thereof a therapeutically effective amount of a pharmaceutical composition

In certain embodiments, a composition comprises mesembranol (Compound 18) or a pharmaceutically acceptable salt thereof and less than about 0.5% of mesembrine or mesembrenone as measured by HPLC. In certain embodiments, a composition comprises (−) 6-epi-mesembranol (Compound 19) or a pharmaceutically acceptable salt thereof and less than about 0.5% of mesembrine or mesembrenone as measured by HPLC.

In certain embodiments, a method of treating anxiety, the method comprises administering to a subject in need thereof a pharmaceutical composition comprising mesembranol (or a pharmaceutically acceptable salt thereof) and/or (−) 6-epi-mesembranol (or a pharmaceutically acceptable salt thereof). In certain embodiments, a method of inhibiting the serotonin transporter (5-HTT) in the central nervous system of a subject, the method comprises the step of administering a pharmaceutical composition comprising mesembranol (or a pharmaceutically acceptable salt thereof) and/or (−) 6-epi-mesembranol (or a pharmaceutically acceptable salt thereof) to a subject in need thereof, wherein the pharmaceutical composition does not inhibit PDE4A1A, PDE4B2, PDE4C1 or PDE4D2 by more than 5% at 10 micromolar.

In some embodiments, mesembranol (Compound 18) or (−) 6-epi-mesembranol (Compound 19) can be used to selectively inhibit the serotonin transporter (5-HTT) while avoiding inhibition of phosphodiesterase-4 (PDE4) at or near levels found in other pharmacologically active alkaloids identified in Sceletium tortuosum extracts. Applicants have discovered that mesembranol (Compound 18) and (−) 6-epi-mesembranol (Compound 19) are potent inhibitors of the serotonin transporter protein (5-HTT) having no more than about 5% inhibition of PDE4A1A, PDE4B2, PDE4C1 and PDE4D2 at 10 micromolar. Compound 18 and Compound 19 surprisingly exhibited notably lower activity against PDE4 than other pharmacologically active alkaloid species identified in Sceletium tortuosum extracts. For example, the alkaloid (+) mesembrine (Compound 2) exhibited about 6 to 23 times greater PDE4 activity compared to Compound 18 or Compound 19, as measured % inhibition of PDE4A1A, PDE4B2, PDE4C1 and PDE4D2 at 10 micromolar. Isolated mesembrenol stereoisomers (Compounds 20, 21, 23, and 24) showed about 3 to 13 times greater PDE4A1A activity and about 3 to 12 times greater PDE4B2 activity compared to Compound 18, as measured % inhibition of PDE4A1A, and PDE4B2 at 10 micromolar. In some embodiments, pharmaceutical compositions comprising an active pharmaceutical ingredient (API) consisting of Compound 18 (or a pharmaceutically acceptable salt thereof) and/or Compound 19 (or a pharmaceutically acceptable salt thereof), where the API does not contain mesembrine and mesembrenone.

In certain embodiments, a pharmaceutical composition comprises (−) mesembranol and/or (−) 6-epi-mesembranol. In certain embodiments, the pharmaceutical composition contains no more than about 10% of an alkaloid selected from (−) mesembrine, mesembrenone, mesembrenol, alone or in combination together. For example, the pharmaceutical composition can contain less than about 10%, less than about 5%, less than about 3%, less than about 2%, or less than about 1% by weight of the combination of alkaloids of (−) mesembrine and mesembrenone, or the combination of mesembrenone and mesembrenol, or the combination of (−) mesembrine, mesembrenone, and mesembrenol. In certain embodiments, the composition is substantially free of (−) mesembrine, mesembrenone, and mesembrenol. For example, the pharmaceutical composition can contain alkaloids and less than about 10%, less than about 5%, less than about 3%, less than about 2%, or less than about 1% by weight of the total alkaloids is a combination of alkaloids of (−) mesembrine and mesembrenone, or the combination of mesembrenone and mesembrenol, or the combination of (−) mesembrine, mesembrenone, and mesembrenol. In certain embodiments, the pharmaceutical composition can contain alkaloids, for example about 0.3 wt % to about 0.5 wt % and mesembrenone and mesembrenol are less than about 60 wt % of the total alkaloid content. In certain embodiments, the pharmaceutical composition can contain alkaloids, for example about 0.3 wt % to about 0.5 wt % and mesembrine is less than about 20 wt % of the total alkaloid content. In certain embodiments, the pharmaceutical composition can contain alkaloids, for example about 0.3 wt % to about 0.5 wt % and (−) mesembranol and/or (−) 6-epimesembranol is at least about 70%, at least 80%, or at least about 90% of the total amount of alkaloid in the composition. In certain embodiments, the composition comprises less than about 70 micrograms of the combination of mesembrenone and mesembrenol. In certain embodiments, the composition comprises less than about 23 micrograms of (−) mesembrine.

In certain embodiments, the composition comprises less than about 70 micrograms of mesembrenone and mesembrenol per 11.3 mg total alkaloid content. In certain embodiments, the composition comprises less than about 23 micrograms of (−) mesembrine per 11.3 mg total alkaloid content

In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety, stress, and depression. In some embodiments, a method comprises the administration to a patient in need thereof of a therapeutically effective amount of a compound selected from (−) mesembranol and/or (−) 6-epi-mesembranol for the treatment of depression. In some embodiments, a method comprises the administration to a patient in need thereof of a therapeutically effective amount of a compound of (−) mesembranol and/or (−) 6-epi-mesembranol for the treatment of a condition selected from the group consisting of: anxiety associated with depression, anxiety with depression, mixed anxiety and depressive disorder. In some embodiments, a method comprises the administration to a patient in need thereof of a therapeutically effective amount of a compound of (−) mesembranol and/or (−) 6-epi-mesembranol for the treatment of anxiety and hysteria or anxiety and depression.

In certain embodiments, the present disclosure provides a method of treating a mental disorder, comprising administering to the subject a compound of the present disclosure (e.g., a compound selected from (−) mesembranol and/or (−) 6-epi-mesembranol).

In certain embodiments, the present disclosure provides a method of inhibiting SERT, comprising administering to a mammal in need thereof a therapeutically effective amount a compound selected from (−) mesembranol and (−) 6-epi-mesembranol. In certain embodiments, the compound is (−) mesembranol. In certain embodiments, the compound is (−) 6-epi-mesembranol. In certain embodiments, the compound (e.g., (−) mesembranol or (−) 6-epi-mesembranol) is in the form of a composition according to the present disclosure.

In certain embodiments, the present disclosure provides a method of inhibiting SERT, comprising administering to a mammal in need thereof a therapeutically effective amount of a pharmaceutical composition comprising a compound selected from (−) mesembranol and (−) 6-epi-mesembranol or a salt thereof. In certain embodiments, the compound is (−) mesembranol. In certain embodiments, the compound is (−) 6-epi-mesembranol. In certain embodiments, pharmaceutical composition contains no more than about 10% of an alkaloid selected from (−) mesembrine, mesembrenone, mesembrenol, alone or in combination together. For example, the pharmaceutical composition can contain less than about 10%, less than about 5%, less than about 3%, less than about 2%, or less than about 1% by weight of the combination of alkaloids of (−) mesembrine and mesembrenone, or the combination of mesembrenone and mesembrenol, or the combination of (−) mesembrine, mesembrenone, and mesembrenol. In certain embodiments, the composition is substantially free of (−) mesembrine, mesembrenone, and mesembrenol.

In some embodiments, the compound disclosed herein is administered to the patient in a unit dose. In some embodiments, the compound disclosed herein is prescribed to a patient in an oral unit dose for such as a capsule or tablet once or more times per day. In some embodiments, a compound disclosed herein is administered to a patient for the treatment of a disease or condition for which mesembranol (e.g., (−) mesembranol or (−) 6-epi-mesembranol) is safe and effective for treatment. In some embodiments, a method comprises the administration to a patient in need thereof of a therapeutically effective amount of a mesembranol (e.g., (−) mesembranol or (−) 6-epi-mesembranol) for the treatment of anxiety. In some embodiments, a method comprises the administration to a patient in need thereof of a therapeutically effective amount of a mesembranol (e.g., (−) mesembranol or (−) 6-epi-mesembranol) for the treatment of a disease selected from the group consisting of mild to moderate depression and major depressive episodes. In some embodiments, a method comprises the administration to a patient in need thereof of a therapeutically effective amount of mesembranol (e.g., (−) mesembranol or (−) 6-epi-mesembranol) for the treatment of a disease selected from the group consisting of psychological and psychiatric disorders where anxiety is present. In some embodiments, a method comprises the administration to a patient in need thereof of a therapeutically effective amount of mesembranol (e.g., (−) mesembranol or (−) 6-epi-mesembranol) for the treatment of major depressive episodes. In some embodiments, a method comprises the administration to a patient in need thereof of a therapeutically effective amount of mesembranol (e.g., (−) mesembranol or (−) 6-epi-mesembranol) for the treatment of a disease selected from the group consisting of alcohol and drug dependence, bulimia nervosa, and obsessive-compulsive disorders. In some embodiments, an amount of from 20 micrograms to 2 milligrams of mesembranol (e.g., (−) mesembranol or (−) 6-epi-mesembranol) is orally administered to a patient to treat the patient in need thereof. In some embodiments, an amount of from 20 micrograms to 2 milligrams of mesembranol (e.g., (−) mesembranol or (−) 6-epi-mesembranol) is orally administered to a patient to treat the patient in need thereof.

In some embodiments, a method comprises the administration to a patient in need thereof of a therapeutically effective amount of mesembranol (e.g., (−) mesembranol or (−) 6-epi-mesembranol) for the treatment of a disease selected from the group consisting of Acute and maintenance treatment of Major Depressive Disorder (MDD), Acute and maintenance treatment of Obsessive Compulsive Disorder (OCD), Acute and maintenance treatment of Bulimia Nervosa, and Acute treatment of Panic Disorder, with or without agoraphobia.

In some embodiments, a method comprises the administration to a patient in need thereof of a therapeutically effective amount of mesembranol (e.g., (−) mesembranol or (−) 6-epi-mesembranol) for the treatment of a disease selected from the group consisting of Major Depressive Disorder (MDD), Obsessive Compulsive Disorder (OCD), Panic Disorder (PD), Social Anxiety Disorder (SAD), Generalized Anxiety Disorder (GAD), and Posttraumatic Stress Disorder (PTSD).

In some embodiments, a method comprises the administration to a patient in need thereof of a therapeutically effective amount of mesembranol (e.g., (−) mesembranol or (−) 6-epi-mesembranol) for the treatment of a disease selected from the group consisting of Major depressive disorder (MDD), Obsessive-compulsive disorder (OCD), Panic disorder (PD), Posttraumatic stress disorder (PTSD), Social anxiety disorder (SAD), and Premenstrual dysphoric disorder (PMDD).

In certain embodiments, the (−) 6-epi-mesembranol of the pharmaceutical composition has a half-life of at least 30 minutes, at least about 60 minutes, at least about 90 minutes, or at least about 120 minutes in a human hepatocyte assay, for example, one performed according to Example 4.

In certain embodiments, the ratio AUCₗₐₛₜ of (−) 6-epi-mesembranol to (−) mesembrine is about 2 to about 2.5, for example, in an assay performed according to Example 5. In certain embodiments, the AUCₗₐₛₜ of (−) mesembranol to (−) mesembrine is about 4 to about 4.5, for example, in an assay performed according to Example 5.

In certain embodiments, Compound 018 has about a 3-fold longer plasma half-life, about a 9-fold higher Cmax, and about a 4-fold greater AUC(last) compared to mesembrine (Compound 001), for example, in an assay performed according to Example 5. In certain embodiments, Compound 019 has about a 2-fold longer plasma half-life, a 2-fold higher Cmax, and about a 2-fold greater AUC(last) compared to mesembrine (Compound 001), for example, in an assay performed according to Example 5.

In certain embodiments, a method of inhibiting SERT without inhibiting PDE4, comprises administering to mammal in need thereof a therapeutically effective amount of a pharmaceutical composition comprising a compound selected from (−) mesembranol and (−) 6-epi-mesembranol. In some embodiments, the pharmaceutical composition has an IC₅₀ for SERT (e.g., as performed in Example 3A) of less than about 30 nM or less than about 15 nM. In certain embodiments, the pharmaceutical composition has a % inhibition (e.g., as performed in Example 3B) of less than about 6% for at least one of PDE4A1A, PDE4B2, PDE4C1, and PDE4D2. In some embodiments, the pharmaceutical composition has a % inhibition (e.g., as performed in Example 3B) of less than about 6% for at least two of PDE4A1A, PDE4B2, PDE4C1, and PDE4D2. In some embodiments, the pharmaceutical composition has a % inhibition (e.g., as performed in Example 3B) of less than about 6% for at least three of PDE4A1A, PDE4B2, PDE4C1, and PDE4D2. In some embodiments, the pharmaceutical composition has a % inhibition (e.g., as performed in Example 3B) of less than about 6% for each of PDE4A1A, PDE4B2, PDE4C1, and PDE4D2.

### Pharmaceutical Compositions

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

In some embodiments, pharmaceutical compositions comprise Compound 18 and/or Compound 19, or pharmaceutically acceptable salts thereof, in the absence of one or more pharmacologically active alkaloid compounds in in Sceletium tortuosum extracts. Pharmaceutical compositions comprising Compound 18, Compound 19 or mixtures thereof, including pharmaceutically acceptable salts of Compound 18 and Compound 19, can be used for inhibiting the serotonin transporter (5-HTT) while avoiding inhibition of phosphodiesterase-4 (PDE4) at levels found in other pharmacologically active alkaloids identified in Sceletium tortuosum extracts, such as mesembrine and mesembrenone.

In some embodiments, a pharmaceutical composition can comprise mesembranol (Compound 18), and/or (−) 6-epi-mesembranol (Compound 19), or pharmaceutically acceptable salts thereof, including mixtures of Compound 18 and Compound 19, with less than 0.5% mesembrine or mesembrenone detectable by HPLC. In some embodiments, a pharmaceutical composition can comprise mesembranol (Compound 18) or pharmaceutically acceptable salts thereof, with less than 0.5% mesembrine or mesembrenone detectable by HPLC. In some embodiments, a pharmaceutical composition can comprise (−) 6-epi-mesembranol (Compound 19) or pharmaceutically acceptable salts thereof, with less than 0.5% mesembrine or mesembrenone detectable by HPLC.

In some embodiments, a pharmaceutical composition comprises an API consisting of Compound 18 (or a pharmaceutically acceptable salt thereof) and/or Compound 19 (or a pharmaceutically acceptable salt thereof) for the treatment of anxiety in a subject in need thereof. In some embodiments, a pharmaceutical composition comprises an API consisting of Compound 18 (or a pharmaceutically acceptable salt thereof) is a selective serotonin reuptake inhibitor for the treatment of anxiety in a subject in need thereof. In some embodiments, a pharmaceutical composition comprises an API consisting of Compound 19 (or a pharmaceutically acceptable salt thereof) for the treatment of anxiety in a subject in need thereof. In some embodiments, a pharmaceutical composition comprises an API consisting of Compound 18 (or a pharmaceutically acceptable salt thereof) and/or Compound 19 (or a pharmaceutically acceptable salt thereof) for the treatment of anxiety in a subject in need thereof, in the absence of mesembrine or mesembrenone.

In some embodiments, a pharmaceutical composition comprises an API consisting of Compound 18 (or a pharmaceutically acceptable salt thereof) and/or Compound 19 (or a pharmaceutically acceptable salt thereof) for the treatment of General Anxiety Disorder (GAD). In some embodiments, a pharmaceutical composition comprises an API consisting of Compound 18 (or a pharmaceutically acceptable salt thereof) for the treatment of General Anxiety Disorder (GAD). In some embodiments, a pharmaceutical composition comprises an API consisting of Compound 19 (or a pharmaceutically acceptable salt thereof) for the treatment of General Anxiety Disorder (GAD). In some embodiments, a pharmaceutical composition comprises an API consisting of Compound 18 (or a pharmaceutically acceptable salt thereof) and/or Compound 19 (or a pharmaceutically acceptable salt thereof) for the treatment of General Anxiety Disorder (GAD) in the absence of mesembrine or mesembrenone.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g. intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In some embodiments, pharmaceutical compositions comprising compounds of Compound 18 and/or Compound 19 or pharmaceutically acceptable salts thereof can be formulated for oral administration. For example, a compound provided herein can be combined with suitable compendial excipients to form an oral unit dosage form, such as a capsule or tablet, containing a target dose of mesembranol. The drug product can be prepared by first manufacturing mesembranol as an active pharmaceutical ingredient (API), followed by roller compaction/milling with intragranular excipients and blending with extra granular excipients. A Drug Product can contain mesembranol as the API and excipient components in a tablet in a desired dosage strength. The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include mesembranol as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present invention may contain conventional pharmaceutical carriers and/or auxiliary agents. In some embodiments, he pharmaceutical compositions according to the present invention may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

Liquid dosage forms useful for oral administration include pharmaceutically acceptable carriers and the active ingredient provided as a solid form for reconstitution prior to administration or as a liquid (e.g., solutions, suspensions, or emulsions). In addition to the active ingredient, a liquid dosage forms may contain inert diluents commonly used in the art. For example, formulations of pharmaceutically acceptable compositions for injection can include aqueous solutions such as water or physiologically buffered saline or other solvents or vehicles suitable for the intended route of administration. In some embodiments, the pharmaceutical composition is formulated for parenteral administration.

The therapeutically effective amount of a pharmaceutical composition can be determined by human clinical trials to determine the safe and effective dose for a patient with a relevant diagnosis. It is generally understood that the effective amount of the compound may vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the pharmaceutical composition at a dose and dose interval determined to be safe and effective for the patient.

In certain embodiments, the pharmaceutical compositions can contain less than about 10%, less than about 5%, less than about 3%, less than about 2%, or less than about 1% by weight of the combination of alkaloids of (−) mesembrine and mesembrenone, or the combination of mesembrenone and mesembrenol, or the combination of (−) mesembrine, mesembrenone, and mesembrenol. In certain embodiments, the composition is substantially free of (−) mesembrine, mesembrenone, and mesembrenol.

In certain embodiments, the pharmaceutical composition comprises a therapeutically effective amount of (−) mesembranol or (−) 6-epi-mesembranol.

The present disclosure includes the use of pharmaceutically acceptable salts of compounds of the invention in the compositions and methods of the present invention. Pharmaceutically acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some embodiments, a pharmaceutically acceptable salt is a metal salt, in some embodiments, a pharmaceutically acceptable salt is an ammonium salt. For example, a pharmaceutically acceptable acid addition salt can exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

In some embodiments, a pharmaceutical composition comprises Compound 018 or a pharmaceutically acceptable salt thereof, and excipients such as contain starch, gelatin, silicone, titanium dioxide, iron oxide, and other inactive ingredients such as coloring dye. In some embodiments, a tablet or capsule contains Compound 018, or a pharmaceutical composition thereof, and the inactive ingredients.

In some embodiments, a pharmaceutical composition comprises Compound 019 or a pharmaceutically acceptable salt thereof, and excipients such as contain starch, gelatin, silicone, titanium dioxide, iron oxide, and other inactive ingredients such as coloring dye. In some embodiments, a tablet or capsule contains Compound 019, or a pharmaceutical composition thereof, and the inactive ingredients.

### Selective Inhibition of 5-HTT

In some embodiments, mesembranol (Compound 18) or a pharmaceutically acceptable salt thereof can be used in the absence of mesembranone and mesembrine to inhibit the serotonin transporter (5-HTT) without inhibiting phosphodiesterase-4 (PDE4). In some embodiments, (−) 6-epi-mesembranol (Compound 19) or a pharmaceutically acceptable salt thereof, can be used in the absence of mesembrine and mesembrenone to inhibit the serotonin transporter (5-HTT) without inhibiting phosphodiesterase-4 (PDE4).

For a particular composition described herein, a ratio of PDE4/SERT inhibition values may be calculated from (i) the SERT inhibition value (e.g., IC₅₀) for the composition determined via the assay described in Example 3A, and (ii) the PDE4 inhibition value (e.g., IC₅₀) for the same composition determined via the assay described in Example 3B. In certain embodiments, the ratio of PDE4/SERT inhibition values is at least 10:1, at least 100:1, or at least 500:1. In some embodiments, a pharmaceutical composition has an IC₅₀ for SERT of less than about 30 nM or less than about 15 nM.

In some embodiments, methods of inhibiting serotonin transporter (5-HTT) in the central nervous system (CNS) of a subject with an active pharmaceutical ingredient (API) having no more than about 5% inhibition of PDE4A1A, PDE4B2, PDE4C1 and PDE4D2 at 10 micromolar are provided, wherein the methods comprise the administration of an API consisting of mesembranol (Compound 18) or a pharmaceutically acceptable salt thereof, (−) 6-epi-mesembranol (Compound 19) or a pharmaceutically acceptable salt thereof, a mixture of Compound 18 and Compound 19 or a pharmaceutically acceptable salt thereof. In some embodiments, a method of inhibiting serotonin transporter (5-HTT) in the central nervous system (CNS) of a subject with an active pharmaceutical ingredient (API) having no more than about 5% inhibition of PDE4A1A, PDE4B2, PDE4C1 and PDE4D2 at 10 micromolar, comprises the administration of an API consisting of mesembranol (Compound 18) or a pharmaceutically acceptable salt thereof. In some embodiments, a method of inhibiting serotonin transporter (5-HTT) in the central nervous system (CNS) of a subject with an active pharmaceutical ingredient (API) having no more than about 5% inhibition of PDE4A1A, PDE4B2, PDE4C1 and PDE4D2 at 10 micromolar, comprises the administration of an API consisting of (−) 6-epi-mesembranol (Compound 19) or a pharmaceutically acceptable salt thereof. In certain embodiments, the API has a % inhibition of less than about 6% for at least one of PDE4A1A, PDE4B2, PDE4C1, and PDE4D2, for at least two of PDE4A1A, PDE4B2, PDE4C1, and PDE4D2, for at least three of PDE4A1A, PDE4B2, PDE4C1, and PDE4D2, or for each of PDE4A1A, PDE4B2, PDE4C1, and PDE4D2.

In some embodiments, a method of treatment comprises the administration of mesembranol (Compound 18), (−) 6-epi-mesembranol (Compound 19) or a pharmaceutically acceptable salt thereof, in the absence of mesembrine or mesembrenone, to inhibit the serotonin transporter (5-HTT) in a subject in need thereof, without inhibiting phosphodiesterase-4 (PDE4). In some embodiments, a method of treatment comprises the administration of mesembranol (Compound 18), or a pharmaceutically acceptable salt thereof, in the absence of mesembrine or mesembrenone, to inhibit the serotonin transporter (5-HTT) in a subject in need thereof, without inhibiting phosphodiesterase-4 (PDE4). In some embodiments, a method of treatment comprises the administration of (−) 6-epi-mesembranol (Compound 19) or a pharmaceutically acceptable salt thereof, in the absence of mesembrine or mesembrenone, to inhibit the serotonin transporter (5-HTT) in a subject in need thereof, without inhibiting phosphodiesterase-4 (PDE4). In certain mesembranol (Compound 18), (−) 6-epi-mesembranol (Compound 19) or a pharmaceutically acceptable salt thereof has a % inhibition of less than about 6% for at least one of PDE4A1A, PDE4B2, PDE4C1, and PDE4D2, for at least two of PDE4A1A, PDE4B2, PDE4C1, and PDE4D2, for at least three of PDE4A1A, PDE4B2, PDE4C1, and PDE4D2, or for each of PDE4A1A, PDE4B2, PDE4C1, and PDE4D2.

In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, the method comprising administering to the subject an API comprising mesembranol (Compound 18), (−) 6-epi-mesembranol (Compound 19) or a pharmaceutically acceptable salt thereof. In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, the method comprising administering to the subject an API comprising mesembranol (Compound 18) or a pharmaceutically acceptable salt thereof. In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, the method comprising administering to the subject an API consisting of mesembranol (Compound 18) or a pharmaceutically acceptable salt thereof. In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, the method comprising administering to the subject an API comprising (−) 6-epi-mesembranol (Compound 19) or a pharmaceutically acceptable salt thereof. In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, the method comprising administering to the subject an API consisting of (−) 6-epi-mesembranol (Compound 19) or a pharmaceutically acceptable salt thereof. In certain embodiments, the API has a % inhibition of less than about 6% for at least one of PDE4A1A, PDE4B2, PDE4C1, and PDE4D2, for at least two of PDE4A1A, PDE4B2, PDE4C1, and PDE4D2, for at least three of PDE4A1A, PDE4B2, PDE4C1, and PDE4D2, or for each of PDE4A1A, PDE4B2, PDE4C1, and PDE4D2.

In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, comprises administering to the subject mesembranol (Compound 18), (−) 6-epi-mesembranol (Compound 19) or a pharmaceutically acceptable salt thereof in the absence of mesembrine or mesembrenone. In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, the method comprising administering to the subject mesembranol (Compound 18) or a pharmaceutically acceptable salt thereof in the absence of mesembrine and mesembrenone. In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, the method comprising administering to the subject mesembranol (Compound 18) or a pharmaceutically acceptable salt thereof in the absence of mesembrine. In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, the method comprising administering to the subject mesembranol (Compound 18) or a pharmaceutically acceptable salt thereof in the absence of mesembrenone. In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, the method comprising administering to the subject (−) 6-epi-mesembranol (Compound 19) or a pharmaceutically acceptable salt thereof in the absence of mesembrine and mesembrenone. In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, the method comprising administering to the subject (−) 6-epi-mesembranol (Compound 19) or a pharmaceutically acceptable salt thereof in the absence of mesembrine. In some embodiments, a method of inhibiting the serotonin transporter (5-HTT) in a subject in need thereof with an active pharmaceutical ingredient (API) having no more than about 5% inhibition of PDE4A1A, PDE4B2, PDE4C1 and PDE4D2 at 10 micromolar, the method comprising administering to the subject (−) 6-epi-mesembranol (Compound 19) or a pharmaceutically acceptable salt thereof in the absence of mesembrenone. In certain embodiments, the API has a % inhibition of less than about 6% for at least one of PDE4A1A, PDE4B2, PDE4C1, and PDE4D2, for at least two of PDE4A1A, PDE4B2, PDE4C1, and PDE4D2, for at least three of PDE4A1A, PDE4B2, PDE4C1, and PDE4D2, or for each of PDE4A1A, PDE4B2, PDE4C1, and PDE4D2.

In some embodiments, methods of treatment are provided, comprising the administration of a pharmaceutical composition comprising an API consisting of Compound 18 (or a pharmaceutically acceptable salt thereof) and/or Compound 19 (or a pharmaceutically acceptable salt thereof).

In some embodiments, methods of treating Generalized Anxiety Disorder are provided. Generalized Anxiety Disorder (DSM-IV) is characterized by excessive anxiety and worry (apprehensive expectation) that is persistent for at least 6 months and which the person finds difficult to control. It must be associated with at least 3 of the following symptoms: restlessness or feeling keyed up or on edge, being easily fatigued, difficulty concentrating or mind going blank, irritability, muscle tension, and sleep disturbance. In some embodiments, a method of treating Generalized Anxiety Disorder (GAD) comprises the administration of a pharmaceutical composition comprising Compound 18 (or a pharmaceutically acceptable salt thereof) as the API. In some embodiments, a method of treating Generalized Anxiety Disorder (GAD) comprises the administration of a pharmaceutical composition comprising Compound 19 (or a pharmaceutically acceptable salt thereof) as the API. In some embodiments, a method of treating Generalized Anxiety Disorder (GAD) comprises the administration of a pharmaceutical composition comprising a mixture of Compound 18 (or a pharmaceutically acceptable salt thereof) and Compound 19 (or a pharmaceutically acceptable salt thereof) as the API. In some embodiments, a method of treating Generalized Anxiety Disorder (GAD) comprises the administration of a pharmaceutical composition comprising Compound 18 (or a pharmaceutically acceptable salt thereof) as the API in the absence of mesembrine or mesembrenone. In some embodiments, a method of treating Generalized Anxiety Disorder (GAD) comprises the administration of a pharmaceutical composition comprising Compound 18 (or a pharmaceutically acceptable salt thereof) as the API, wherein the pharmaceutical composition contains 0-0.5% mesembrine or mesembrenone as measured by HPLC. In some embodiments, a method of treating Generalized Anxiety Disorder (GAD) comprises the administration of a pharmaceutical composition comprising Compound 19 (or a pharmaceutically acceptable salt thereof) as the API, wherein the pharmaceutical composition contains 0-0.5% mesembrine or mesembrenone as measured by HPLC. In some embodiments, a method of treating Generalized Anxiety Disorder (GAD) comprises the administration of a pharmaceutical composition comprising a mixture of Compound 18 (or a pharmaceutically acceptable salt thereof) and Compound 19 (or a pharmaceutically acceptable salt thereof) as the API, wherein the pharmaceutical composition contains 0-0.5% mesembrine or mesembrenone as measured by HPLC.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines), companion animals (e.g., canines, felines) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds of a compound described herein.

Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. The mono- or di-acid salts can be formed, and such salts may exist in either a hydrated, solvated or substantially anhydrous form. In general, the acid addition salts of compounds as described herein are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds as described herein for laboratory use, or for subsequent conversion to a pharmaceutically acceptable acid addition salt.

The term “pharmaceutically acceptable basic addition salt” as used herein means any non-toxic organic or inorganic base addition salt of any acid compounds as described herein or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### Additional Embodiments

1. A method of treating a mental health disorder, comprising administering to a mammal in need thereof an effective amount of a compound selected from (−) mesembranol and (−) 6-epi-mesembranol, or a pharmaceutically acceptable salt thereof.

2. The method of embodiment 1, wherein the compound is (−) mesembranol.

3. The method of embodiment 1, wherein the compound is (−) 6-epi-mesembranol.

4. The method of any one of embodiments 1-3, wherein the mental health disorder is anxiety, stress, or depression.

5. The method of embodiment 4, wherein the mental health disorder is anxiety.

6. The method of embodiment 4, wherein the mental health disorder is stress.

7. The method of embodiment 4, wherein the mental health disorder is depression.

8. The method of any one of embodiments 1-7, wherein the mammal is a human.

9. A method of inhibiting SERT, comprising administering to a mammal in need thereof a therapeutically effective amount of a compound selected from (−) mesembranol and (−) 6-epi-mesembranol; or a pharmaceutically acceptable salt thereof.

10. The method of embodiment 9, wherein the compound is (−) mesembranol.

11. The method of embodiment 9, wherein the compound is (−) 6-epi-mesembranol.

12. A method of inhibiting SERT, comprising administering to a mammal in need thereof a therapeutically effective amount of a pharmaceutical composition comprising a compound selected from (−) mesembranol and (−) 6-epi-mesembranol, or a pharmaceutically acceptable.

13. The method of embodiment 12, wherein the compound is (−) mesembranol.

14. The method of embodiment 12, wherein the compound is (−) 6-epi-mesembranol.

15. The method of any one of embodiments 12-14, wherein the pharmaceutical composition contains no more than about 10% of an alkaloid selected from (−) mesembrine, mesembrenone, mesembrenol, or a combination thereof.

16. The method of any one of embodiments 12-14, wherein the pharmaceutical composition contains no more than about 5% of an alkaloid selected from (−) mesembrine, mesembrenone, mesembrenol, or a combination thereof.

17. The method of any one of embodiments 12-14, wherein the pharmaceutical composition contains no more than about 3% of an alkaloid selected from (−) mesembrine, mesembrenone, mesembrenol, or a combination thereof.

18. The method of any one of embodiments 12-14, wherein the pharmaceutical composition contains no more than about 2% of an alkaloid selected from (−) mesembrine, mesembrenone, mesembrenol, or a combination thereof.

19. The method of any one of embodiments 12-14, wherein the pharmaceutical composition contains no more than about 1% of an alkaloid selected from (−) mesembrine, mesembrenone, mesembrenol, or a combination thereof.

20. The method of any one of embodiments 15-19, wherein the alkaloid is (−) mesembrine.

21. The method of any one of embodiments 15-19, wherein the alkaloid is mesembrenone.

22. The method of any one of embodiments 15-19, wherein the alkaloid is mesembrenol.

23. The method of any one of embodiments 15-19, wherein the pharmaceutical composition is substantially free of (−) mesembrine, mesembrenone, and mesembrenol.

24. A pharmaceutical composition, comprising (−) 6-epi-mesembranol and a pharmaceutically acceptable excipient.

25. The pharmaceutical composition of embodiment 24, wherein the pharmaceutical composition contains no more than about 10% of an alkaloid selected from (−) mesembrine, mesembrenone, mesembrenol, or a combination thereof.

26. The pharmaceutical composition of embodiment 24, wherein the pharmaceutical composition contains no more than about 5% of an alkaloid selected from (−) mesembrine, mesembrenone, mesembrenol, or a combination thereof.

27. The pharmaceutical composition of embodiment 24, wherein the pharmaceutical composition contains no more than about 3% of an alkaloid selected from (−) mesembrine, mesembrenone, mesembrenol, or a combination thereof.

28. The pharmaceutical composition of embodiment 24, wherein less than about 60% of the total alkaloid content is mesembrenone and mesembrenol.

29. The pharmaceutical composition of embodiment 24, wherein less than about 20% of the total alkaloid content is (−) mesembrine.

30. The pharmaceutical composition of embodiment 24, wherein the composition comprises about 0.3 wt % to about 0.5 wt % total alkaloids; and mesembrenone and mesembrenol are less than about 60 wt % of the total alkaloid content.

31. The pharmaceutical composition of embodiment 24, wherein the composition comprises about 0.3 wt % to about 0.6 wt % total alkaloids and mesembrine is less than about 20 wt % of the total alkaloid content.

32. The pharmaceutical composition of embodiment 30 or 31, wherein (−) 6-epi-mesembranol is at least 70% of the total alkaloid content in the composition.

33. The pharmaceutical composition of embodiment 30 or 31, wherein (−) 6-epi-mesembranol is at least 80% of the total alkaloid content in the composition.

34. The pharmaceutical composition of embodiment 30 or 31, wherein (−) 6-epi-mesembranol is at least 90% of the total alkaloid content in the composition.

35. The pharmaceutical composition of embodiment 24, wherein the composition comprises less than about 70 micrograms of mesembrenone and mesembrenol per 11.3 mg total alkaloid content.

36. The pharmaceutical composition of embodiment 24, wherein the composition comprises less than about 23 micrograms of (−) mesembrine per 11.3 mg total alkaloid content.

37. The pharmaceutical composition of any one of embodiments 24-36, wherein the ratio of PDE4/SERT inhibition values is at least 10:1.

38. The pharmaceutical composition of any one of embodiments 24-36, wherein the ratio of PDE4/SERT inhibition values is at least 100:1.

39. The pharmaceutical composition of any one of embodiments 24-36, wherein the ratio of PDE4/SERT inhibition values is at least 500:1.

40. The pharmaceutical composition of any one of embodiments 24-39, wherein the (−) 6-epi-mesembranol has a half-life of at least 30 minutes in a human hepatocyte assay of the pharmaceutical composition.

41. The pharmaceutical composition of any one of embodiments 24-39, wherein the (−) 6-epi-mesembranol has a half-life of at least 60 minutes in a human hepatocyte assay of the pharmaceutical composition.

42. The pharmaceutical composition of any one of embodiments 24-39, wherein the (−) 6-epi-mesembranol has a half-life of at least 90 minutes in a human hepatocyte assay of the pharmaceutical composition.

43. The pharmaceutical composition of any one of embodiments 24-39, wherein the (−) 6-epi-mesembranol has a half-life of at least 120 minutes in a human hepatocyte assay of the pharmaceutical composition.

44. The pharmaceutical composition of any one of embodiments 24-43, wherein the AUCiast ratio of (−) 6-epi-mesembranol to (−) mesembrine is about 2 to about 2.5.

45. A pharmaceutical composition, comprising (−) mesembranol and a pharmaceutically acceptable excipient.

46. The pharmaceutical composition of embodiment 45, wherein the pharmaceutical composition contains no more than about 10% of an alkaloid selected from (−) mesembrine, mesembrenone, mesembrenol, or a combination thereof.

47. The pharmaceutical composition of embodiment 46, wherein the pharmaceutical composition contains no more than about 5% of an alkaloid selected from (−) mesembrine, mesembrenone, mesembrenol, or a combination thereof.

48. The pharmaceutical composition of embodiment 46, wherein the pharmaceutical composition contains no more than about 3% of an alkaloid selected from (−) mesembrine, mesembrenone, mesembrenol, or a combination thereof.

49. The pharmaceutical composition of embodiment 46, wherein the composition contains less than about 60% of mesembrenone and mesembrenol.

50. The pharmaceutical composition of embodiment 46, wherein the composition contains less than about 20% of (−) mesembrine.

51. The pharmaceutical composition of embodiment 46, wherein the composition comprises about 0.3 wt % to about 0.5 wt % total alkaloids and mesembrenone and mesembrenol are less than about 60 wt % of the total alkaloid content.

52. The pharmaceutical composition of embodiment 46, wherein the composition comprises about 0.3 wt % to about 0.6 wt % total alkaloids and mesembrine is less than about 20 wt % of the total alkaloid content.

53. The pharmaceutical composition of embodiment 51 or 52, wherein (−) mesembranol is at least 70% of the total amount of alkaloid in the composition.

54. The pharmaceutical composition of embodiment 51 or 52, wherein (−) mesembranol is at least 80% of the total alkaloid content in the composition.

55. The pharmaceutical composition of embodiment 51 or 52, wherein (−) mesembranol is at least 90% of the total alkaloid content in the composition.

56. The pharmaceutical composition of embodiment 46, wherein the composition comprises less than about 70 micrograms of mesembrenone and mesembrenol per 11.3 mg total alkaloid content.

57. The pharmaceutical composition of embodiment 46, wherein the composition comprises less than about 23 micrograms of (−) mesembrine per 11.3 mg total alkaloid content.

58. The pharmaceutical composition of any one of embodiments 45-57, wherein the ratio of PDE4/SERT inhibition values is at least 10:1.

59. The pharmaceutical composition of any one of embodiments 45-57, wherein the ratio of PDE4/SERT inhibition values is at least 100:1.

60. The pharmaceutical composition of any one of embodiments 45-57, wherein the ratio of PDE4/SERT inhibition values is at least 500:1.

61. The pharmaceutical composition of any one of embodiments 45-60, wherein the (−) 6-epi-mesembranol has a half-life of at least 30 minutes in a human hepatocyte assay of the pharmaceutical composition.

62. The pharmaceutical composition of any one of embodiments 45-60, wherein the (−) 6-epi-mesembranol has a half-life of at least 60 minutes in a human hepatocyte assay of the pharmaceutical composition.

63. The pharmaceutical composition of any one of embodiments 45-60, wherein the (−) 6-epi-mesembranol has a half-life of at least 90 minutes in a human hepatocyte assay of the pharmaceutical composition.

64. The pharmaceutical composition of any one of embodiments 45-60, wherein the (−) 6-epi-mesembranol has a half-life of at least 120 minutes in a human hepatocyte assay of the pharmaceutical composition.

65. The pharmaceutical composition of any one of embodiments 45-64, wherein the AUCₗₐₛₜratio of (−) 6-epi-mesembranol to (−) mesembrine is about 2 to about 2.5.

66. A method of inhibiting SERT without inhibiting PDE4, comprising administering to mammal in need thereof a therapeutically effective amount of a pharmaceutical composition according to any one of embodiments 24-65.

67. The method of embodiment 66, wherein the pharmaceutical composition comprises (−) mesembranol.

68. The method of embodiment 66, wherein the pharmaceutical composition comprises (−) 6-epi-mesembranol.

69. The method of any one of embodiments 66-68, wherein the pharmaceutical composition has an IC50 for SERT of less than about 30 nM.

70. The method of embodiment 66 or 68, wherein the pharmaceutical composition has an IC₅₀ for SERT of less than about 15 nM.

71. The method of any one of embodiments 66-70, wherein the pharmaceutical composition has a % inhibition of less than about 6% for at least one of PDE4A1A, PDE4B2, PDE4C1, and PDE4D2.

72. The method of any one of embodiments 66-70, wherein the pharmaceutical composition has a % inhibition of less than about 6% for at least two of PDE4A1A, PDE4B2, PDE4C1, and PDE4D2.

73. The method of any one of embodiments 66-70, wherein the pharmaceutical composition has a % inhibition of less than about 6% for at least three of PDE4A1A, PDE4B2, PDE4C1, and PDE4D2.

74. The method of any one of embodiments 66-70, wherein the pharmaceutical composition has a % inhibition of less than about 6% for each of PDE4A1A, PDE4B2, PDE4C1, and PDE4D2.

75. The method of any one of embodiments 66-74 wherein the ratio of PDE4/SERT inhibition values is at least 10:1.

76. The method of any one of embodiments 66-74, wherein the ratio of PDE4/SERT inhibition values is at least 100:1.

77. The method of any one of embodiments 66-74, wherein the ratio of PDE4/SERT inhibition values is at least 500:1.

78. A method of inhibiting SERT without inhibiting PDE4, comprising administering to mammal in need thereof a therapeutically effective amount of a pharmaceutical composition.

79. The method of embodiment 78, wherein the pharmaceutical composition is a composition according to any one of embodiments 24-65.

80. The method embodiment 78 or 79, wherein the pharmaceutical composition has an IC₅₀ for SERT of less than about 30 nM.

81. The method of embodiment 78 or 79, wherein the pharmaceutical composition has an IC₅₀ for SERT of less than about 15 nM.

82. The method of any one of embodiments 78-81, wherein the pharmaceutical composition has a % inhibition of less than about 6% for at least one of PDE4A1A, PDE4B2, PDE4C1, and PDE4D2.

83. The method of any one of embodiments 78-81, wherein the pharmaceutical composition has a % inhibition of less than about 6% for at least two of PDE4A1A, PDE4B2, PDE4C1, and PDE4D2.

84. The method of any one of embodiments 78-81, wherein the pharmaceutical composition has a % inhibition of less than about 6% for at least three of PDE4A1A, PDE4B2, PDE4C1, and PDE4D2.

85. The method of any one of embodiments 78-81, wherein the pharmaceutical composition has a % inhibition of less than about 6% for each of PDE4A1A, PDE4B2, PDE4C1, and PDE4D2.

86. The method of any one of embodiments 78-85, wherein the ratio of PDE4/SERT inhibition values is at least 10:1.

87. The method of any one of embodiments 78-85 wherein the ratio of PDE4/SERT inhibition values is at least 100:1.

88. The method of any one of embodiments 78-85, wherein the ratio of PDE4/SERT inhibition values is at least 500:1.

89. A composition comprising mesembranol (Compound 18) or a pharmaceutically acceptable salt thereof; wherein the composition comprises less than about 0.5% of mesembrine or mesembrenone as measured by HPLC.

90. The composition of embodiment 89, further comprising (−) 6-epi-mesembranol (Compound 19) or a pharmaceutically acceptable salt thereof.

91. A composition comprising (−) 6-epi-mesembranol (Compound 19) or a pharmaceutically acceptable salt thereof; wherein the composition comprises less than about 0.5% of mesembrine or mesembrenone as measured by HPLC.

92. The composition of any one of embodiments 89-91, wherein the composition does not contain mesembrine or mesembrenone.

93. The composition of any one of embodiments 89-92, wherein the composition does not contain an additional alkaloid compound other than Compound 18 or Compound 19.

94. The composition of embodiment 89, wherein the composition is a pharmaceutical composition comprising an active pharmaceutical ingredient (API) consisting of Compound 18 (or a pharmaceutically acceptable salt thereof).

95. The composition of embodiment 91, wherein the composition is a pharmaceutical composition comprising an active pharmaceutical ingredient (API) consisting of Compound 19 (or a pharmaceutically acceptable salt thereof).

96. The composition of any one of embodiments 89-93, wherein the composition is a pharmaceutical composition comprising an active pharmaceutical ingredient (API) consisting of Compound 18 (or a pharmaceutically acceptable salt thereof) and/or Compound 19 (or a pharmaceutically acceptable salt thereof).

97. A method of treating anxiety, the method comprising administering to a subject in need thereof a pharmaceutical composition comprising mesembranol (or a pharmaceutically acceptable salt thereof) and/or (−) 6-epi-mesembranol (or a pharmaceutically acceptable salt thereof).

98. The method of embodiment 97, wherein the active pharmaceutical ingredient (API) of the pharmaceutical composition consists of a mixture of mesembranol (or a pharmaceutically acceptable salt thereof) and/or (−) 6-epi-mesembranol (or a pharmaceutically acceptable salt thereof).

99. The method of embodiment 97, wherein the active pharmaceutical ingredient (API) of the pharmaceutical composition consists of mesembranol (or a pharmaceutically acceptable salt thereof).

100. The method of embodiment 97, wherein the active pharmaceutical ingredient (API) of the pharmaceutical composition consists of a mixture of (−) 6-epi-mesembranol (or a pharmaceutically acceptable salt thereof).

101. The method of any one of embodiments 97-100, wherein the pharmaceutical composition does not comprise mesembrine.

102. The method of any one of embodiments 97-101, wherein the pharmaceutical composition does not comprise mesembrenone.

103. The method of any one of embodiments 97-100, wherein the pharmaceutical composition contains less than 0.5% mesembrine or mesembrenone in the pharmaceutical composition as detected by HPLC.

104. The method of any one of embodiments 97-103, wherein the method is a method of treating General Anxiety Disorder (GAD) in a subject diagnosed with GAD.

105. A method of inhibiting the serotonin transporter (5-HTT) in the central nervous system of a subject, comprising the step of administering to a subject in need thereof an effective amount of a pharmaceutical composition comprising mesembranol (or a pharmaceutically acceptable salt thereof) and/or (−) 6-epi-mesembranol (or a pharmaceutically acceptable salt thereof), wherein the pharmaceutical composition does not inhibit PDE4A1A, PDE4B2, PDE4C1 or PDE4D2 by more than 5% at 10 micromolar.

106. The method of embodiment 105, wherein the pharmaceutical composition does not comprise mesembrine.

107. The method of any one of embodiments 105-106, wherein the pharmaceutical composition does not comprise mesembrenone.

108. The method of embodiment 105, wherein the pharmaceutical composition comprises less than 0.5% mesembrine or mesembrenone as detected by HPLC.

### Examples

LC/MS spectra were obtained using Agilent 1200\G1956A or SHIMADZU LCMS-2020. Standard LC/MS conditions were as follows (running time 1.55 minutes):

Acidic condition: Mobile Phase A: 0.0375% TFA in water (v/v). Mobile Phase B: 0.01875% TFA in acetonitrile (v/v); Column: Kinetex EVO C18 30*2.1 mm, 5 μm.

Basic condition: Mobile Phase A: 0.025% NH₃·H₂O in water (v/v). Mobile Phase B: Acetonitrile; Column: Kinetex EVO C18 2.1X30 mm, 5 μm.

5-95AB_0.8 min

| Instrument | | SHIMADZU LCMS-2020; | | |
|---|---|---|---|---|
| Software | | LabSolution Version 5.97SP1 | | |
| HPLC | Column | Kinetex ® EVO C18 2.1 × 30 mm 5 um | | |
| | Mobile Phase | A: 0.0375% TFA in water (v/v) | | |
| | | B: 0.01875% TFA in Acetonitrile (v/v) | | |
| | | Time(min) | B(%) | Flow(mL/min) |
| | Gradient | 0.00 | 5.0 | 2.0 |
| | | 0.60 | 95.0 | 2.0 |
| | | 0.78 | 95.0 | 2.0 |
| | | 0.79 | 5.0 | 2.0 |
| | | 0.80 | 5.0 | 2.0 |

| | | |
|---|---|---|
| | Column Temp | 50° C. |
| | Detector | PDA (220 nm&254 nm) |
| MS | Ionization source | ESI |
| | Drying Gas | N2 |
| | Drying Gas Flow | 15(L/min) |
| | DL Voltage | 120 (v) |
| | Qarray DC Voltage | 20 (V) |
| | MS Polarity | Positive |
| | MS Mode | Scan |
| | Mass range | 100-1000 |

Table of Abbreviations

| | |
|---|---|
| Ac | Acetyl |
| ACN | Acetonitrile |
| br | Broad |
| C. | Celsius |
| δ | Chemical shift |
| d | Doublet |
| DCM | Dichloromethane |
| de | Diastereomeric excess |
| DIBAL—H | Diisobutyl aluminum hydride |
| DMAP | 4-Dimethylaminopyridine |
| DMF | N,N-Dimethyformamide |
| ESI | Electrospray ionization |
| Et | Ethyl |
| FA | Formic acid |
| HPLC | High performance liquid chromatography |
| hr | Hours |
| Hz | Hertz |
| IPA | Isopropyl alcohol |
| J | Coupling constant |
| LC-MS | Liquid chromatography-mass spectrometry |
| m | Multiplet |
| M | Molar |
| m/z | Mass to charge ratio |
| Me | Methyl |
| min | Minutes |
| mol | Moles |
| NMR | Nuclear magnetic resonance |
| Psi | pound-force per square inch |
| Pr | propyl |
| q | Quartet |
| s | Singlet |
| t | Triplet |
| t-Bu | Tertiary butyl |
| TEA | Triethylamine |
| THF | Tetrahydrofuran |
| TMS | Trimethylsilyl |

Summary of Mesembranol Compound Designations

| Compound | (−) enantiomer |
|---|---|
| Mesembranol | Compound 018 |
| 6-epi-mesembranol | Compound 019 |

### Example 1: Synthesis of (3aS,6R,7aS)-3a-(3,4-dimethoxyphenyl)-1-methyloctahydro-1H-indol-6-ol (018, (−)-mesembranol)

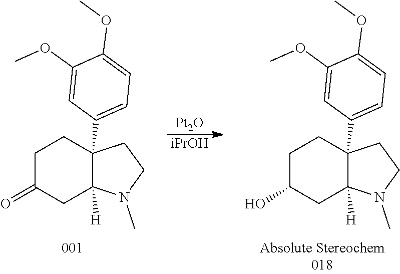

A mixture of 001 (200 mg, 691 umol) and PtO₂ (20.0 mg, 88.0 umol) in IPA (4 mL) was degassed and purged with N₂ 3 times. The mixture was allowed to stir at 25° C. for 16 hr under an atmosphere of N₂. The reaction mixture was filtered and concentrated under reduced pressure. The residue was purified by re-crystallization from EtOH (1 mL) at 25° C. to give (3aS,6R,7aS)-3a-(3,4-dimethoxyphenyl)-1-methyloctahydro-1H-indol-6-ol (018, (−)-mesembranol) (100 mg, 49%) as a white solid. Melting point 145.5-146.5° C. LC-MS (ESI⁺) m/z 292.4 (M+H). ¹H NMR (400 MHz, CDCl₃) δ 6.86-6.78 (m, 2H), 6.77-6.71 (m, 1H), 3.86 (s, 1H), 3.81 (d, J=6.8 Hz, 6H), 3.30 (dt, J=6.8, 9.6 Hz, 1H), 2.83 (s, 1H), 2.40 (s, 3H), 2.33-2.20 (m, 1H), 2.09 (dd, J=2.8, 14.8 Hz, 1H), 1.90-1.82 (m, 2H), 1.78 (dd, J=6.8, 11.6 Hz, 1H), 1.67-1.62 (m, 2H), 1.57 (td, J=2.8, 14.8 Hz, 1H), 1.39-1.30 (m, 2H).

### Example 2: Synthesis of (3aS,6S,7aS)-3a-(3,4-dimethoxyphenyl)-1-methyloctahydro-1H-indol-6-ol (019, (−)-6-epi-mesembranol)

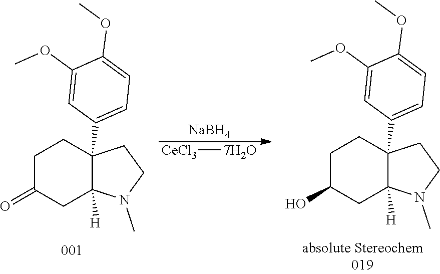

To a solution of 001 (2.00 g, 6.91 mmol) and CeCl₃··7H₂O (3.09 g, 8.29 mmol, 788 uL) in MeOH (80 mL) was added NaBH₄ (1.57 g, 41.4 mmol). The mixture was allowed to stir at 0° C. for 2 hr. The reaction mixture was added into 50 mL NH₄Cl aqueous solution, the organic and aqueous layers were separated, and the aqueous solution was extracted with DCM (50 mL×3). The organic solutions were combined, dried over anhydrous Na₂SO₄, filtered and concentrated under reduced pressure. The residue was purified by prep-HPLC (column: Welch Xtimate C18 150*25 mm*5 um; mobile phase: [water (NH₃H₂O)-ACN]; B %: 28%-58%, 8 min) to give (3aS,6S,7aS)-3a-(3,4-dimethoxyphenyl)-1-methyloctahydro-1H-indol-6-ol (019, (−)-6-epimesembranol) (730 mg, 37%) as white oil. ¹H NMR (400 MHz, CDCl₃) δ 6.95-6.88 (m, 2H), 6.86-6.80 (m, 1H), 3.95 (s, 1H), 3.90 (d, J=6.8 Hz, 6H), 3.46-3.35 (m, 1H), 2.93 (s, 1H), 2.50 (s, 3H), 2.45-2.29 (m, 2H), 2.19 (dd, J=2.4, 14.9 Hz, 1H), 2.01-1.82 (m, 2H), 1.79-1.72 (m, 1H), 1.70-1.59 (m, 3H), 1.44 (tt, J=2.8, 13.6 Hz, 1H).

### Assay Procedures

### Example 3A: SERT Inhibition Assay

SERT inhibition was measured using a Neruotransmitter Transportation Fluorescence assay. Briefly, stable 5HTT HEK293 cells were prepared in a 384 microwell plate (20,000 cells per 20 μL well). Compounds were prepared by in assay buffer (20 mM HEPES in HBSS, 0.1% BSA) at a top concentration of 1 μM. 10 doses of test compound (3-fold serial dilution) were added to the plated cells and incubated for 30 minutes at 37° C. 25 μL of dye solution (Molecular Devices Neurotransmitter Transporter Uptake Assay Kit) was added per well and incubated for 30 minutes at 37° C. The plates were then read on a plate reader and the results are shown in Table 1 (n=6±SD).

TABLE 1

| Compound Name | Compound ID | SERT IC₅₀ (nM) |
|---|---|---|
| (−)-mesembranol | 018 | 21.9 ± 8.0 |
| (−)-6-epimesembranol | 019 | 10.6 ± 10.1 |

### Example 3B: PDE4 Inhibition Assay

### Assay Conditions for PDE4 Percent Inhibition Determination:

100 μM dilutions of the test compounds were prepared in assay buffer (10% DMSO concentration) and 5 μl of the dilution was added to a 50 μl reaction so that the final concentration of DMSO is 1% in all of reactions. The enzymatic reactions were conducted at room temperature for 60 minutes in a 50 μl mixture containing PDE assay buffer, 100 nM FAM-cAMP, a PDE enzyme (Table 2.3.1) and the test compound (Section 2.2). After the enzymatic reaction, 100 μl of a binding solution (1:100 dilution of the binding agent with the binding agent diluent) was added to each reaction and the reaction was performed at room temperature for 60 minutes.
Fluorescence intensity was measured at an excitation of 485 nm and an emission of 528 nm using a Tecan Infinite M1000 microplate reader.

### PDE4 Assay Kit (BPS Bioscience, San Diego, CA), as Described Below.

### Step 1:

1) Dilute 20 μM FAM-Cyclic-3′, 5′-AMP stock 100-fold with PDE buffer to make a 200 nM solution.
2) Add 25 μl of FAM-Cyclic-3′,5′-AMP (200 nM) to each well designated “Positive Control”, “Test Inhibitor”, and “Substrate Control”.
3) Add 20 μl of PDE assay buffer to each well designated “Substrate Control” and 45 μl of PDE assay buffer to each well designated “Blank”.
4) Add 5 μl of inhibitor solution to each well designated “Test Inhibitor”. For the wells labeled “Positive Control”, “Substrate Control” and “Blank”, add 5 μl of the same solution without inhibitor (inhibitor buffer).
5) Thaw PDE on ice. Upon first thaw, briefly spin tube containing enzyme to recover the full contents of the tube.
6) Dilute PDE4 in PDE buffer to 7.5 pg/μl (0.15 ng/reaction)*. Initiate reaction by adding 20 l of PDE4 (7.5 pg/μl) to the wells designated “Positive Control” and “Test Inhibitor.”
7) Incubate at room temperature for 1 hour.

### Step 2:

1) Mix binding agent thoroughly and dilute binding agent 1:100 with binding agent diluent.
2) Add 100 μl diluted binding agent to each microwell. Incubate at room temperature for 1 hour with slow shaking.
3) Read the fluorescent polarization of the sample in a microtiter-plate reader equipped for the measurement of fluorescence polarization, capable of excitation at wavelengths ranging from 485±5 nm and detection of emitted light ranging from 528±10 nm. Blank value is subtracted from all other values.
4) Data Analysis: PDE activity assays were performed in duplicate at each concentration. Fluorescence intensity is converted to fluorescence polarization using the Tecan Magellan6 software. The fluorescence polarization data were analyzed using the computer software, Graphpad Prism. The fluorescence polarization (FPₜ) in absence of the compound in each data set was defined as 100% activity. In the absence of PDE and the compound, the value of fluorescent polarization (FP_{b}) in each data set was defined as 0% activity. The percent activity in the presence of the compound was calculated according to the following equation: % activity=(FP−FP_{b})/(FPₜ−FP_{b})×100%, where FP=the fluorescence polarization in the presence of the compound. The values of % activity versus a series of compound concentrations were then plotted using non-linear regression analysis of sigmoidal dose-response curve generated with the equation Y=B+(T−B)/1+10^{(LogEC50-X)×Hill Slope)}, where Y=percent activity, B=minimum percent activity, T=maximum percent activity, X=logarithm of compound and Hill Slope=slope factor or Hill coefficient. The IC₅₀ value was determined by the concentration causing a half-maximal percent activity. Results are shown in Tables 2A and 2B.

TABLE 2A

| | Com- | PDE4A1A | PDE4B2 | PDE4C1 | PDE4D2 |
|---|---|---|---|---|---|
| Compound | pound | % INH @ | % INH @ | % INH @ | %INH @ |
| Name | ID | 10 μM | 10 μM | 10 μM | 10 μM |
| (−) Mesembranol | 018 | 5 | 3 | 1 | 4 |
| (−) 6-Epi- | 019 | 4 | 0 | 3 | 3 |
| mesembranol | | | | | |
| (+) Mesembrine | 002 | 56 | 60 | 26 | 54 |
| (S, S, R) | 024 | 45 | 55 | 18 | 53 |
| Mesembrenol | | | | | |
| (R, S, R) | 023 | 15 | 16 | 9 | 21 |
| Mesembrenol | | | | | |
| (R, R, S) | 021 | 22 | 12 | 7 | 15 |
| Mesembrenol | | | | | |
| (S, R, S) | 020 | 68 | 62 | 32 | 72 |
| Mesembrenol | | | | | |

TABLE 2B

| Compound | Compound | PDE4A1A | PDE4B2 | PDE4C1 | PDE4D2 |
|---|---|---|---|---|---|
| Name | ID | IC₅₀ (μM) | IC₅₀ (μM) | IC₅₀ (μM) | IC₅₀ (μM) |
| (−) | 001 | 4.3 | 3.2 | 15 | 2.7 |
| Mesembrine | | | | | |

### Example 4: Hepatocyte Metabolism

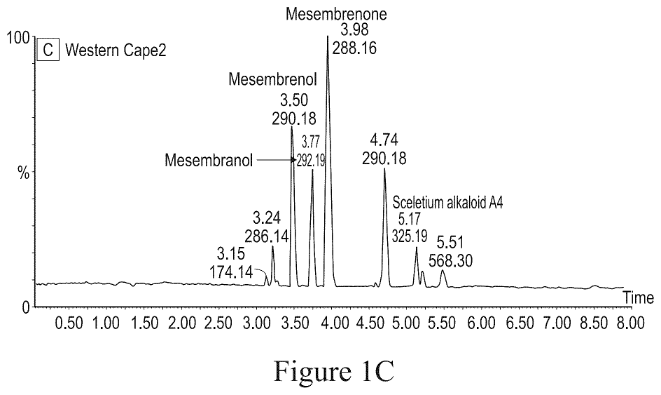

FIG. 2A is a graph showing the compound stability of (−) mesembrine (triangles) compared to (−) mesembranol (squares) in human hepatocytes, measured according to Example 4. FIG. 2B is a graph showing the compound stability of (−) mesembrine (triangles) compared to (−) 6-epi-mesembranol (circles) in human hepatocytes, measured according to Example 4.

A 2.5 uL aliquot of 100 μM test compound was incubated with 247.5 uL of cryopreserved hepatocytes suspended in serum-free incubation medium at 1 million viable cells/mL. The mixture was incubated at 37° C. and shaken at 500 rpm for the designated time points (0.5, 5, 10, 15, 30, 60, 90, or 120 min). At each time point, 25 uL aliquot of incubation mixture was transferred to 125 uL of cold acetonitrile containing internal standard, followed by centrifugation for 30 min at 3,220 g. 100 uL of supernatant was mixed with 100 uL of distilled water for analysis by LC-MS/MS. Peak area ratios were determined from extracted ion chromatograms and percent recovery was calculated. In vitro half-life (t_{1/2}) was determined from the concentration vs time regression slope, in vitro t_{1/2}=0.693/k. Conversion of the in vitro t_{1/2} (in min) into the in vitro intrinsic clearance (in vitro CLᵢₙₜ, in μL/min/10⁶ cells) is done using: in vitro CLᵢₙₜ=k/N. Results are also shown in FIGS. 2A and 2B.

TABLE 3

| | | | Hepatocyte | CLᵢₙₜ | CL_{HB} | | |
|---|---|---|---|---|---|---|---|
| | | | Clᵢₙₜ | (mL/ | (mL/ | | |
| Com- | | t_{1/2} | (μL/min/ | min/ | min/ | % | Extraction |
| pound | Species | (min) | 10⁶ cells) | kg) | kg) | QH | Ratio |
| 001 | Human | 13 | 55 | 131 | 18 | 86 | 0.86 |
| 018 | Human | >120 | 1 | 3 | 3 | 13 | 0.13 |
| 019 | Human | >120 | <1 | <2 | <2 | <10 | <0.10 |

In human hepatocytes, mesembrine (001) undergoes rapid metabolism (t_{1/2}=13 min). In comparison, both mesembranol (018) and 6-epi-mesembranol (019) are highly stable in human hepatocytes (t_{1/2}>120 min for each). These data demonstrate that an oral dose of mesembrine would undergo rapid first-pass hepatic metabolism in vivo, resulting in low concentrations of drug in plasma and brain. In addition, remaining mesembrine concentrations in plasma and brain would be cleared quickly, limiting duration of action. In contrast, oral delivery of mesembranol (018) and 6-epi-mesembranol (019) may have low hepatic clearance in humans and stable drug concentrations may be achieved, allowing for a long duration of action suitable for therapeutic benefit.

In vitro intrinsic clearance data in human hepatocytes is a predictor of a drug's plasma clearance in vivo (drug stability). When the extraction ratio for a given compound is high (highest theoretical=1.0), the drug will be cleared quickly from the body by the liver. Compound 001 has a high extraction ratio (E˜0.9) while compounds 018 and 019 have a low extraction ratio (E˜0.1). As a predictor of drug clearance from plasma in vivo, these data demonstrate 001 would be rapidly cleared in human plasma in vivo.

### Example 5: Pharmacokinetic Studies of Mesembranol, 6-Epi-Mesembranol and Mesembrine Following PO Administration in Male SD Rat

Three separate pharmacokinetic studies were conducted for each compound. In general, 10 mg/kg of compound was administered PO to male SD rats (formulated in 1% NMP and 99% saline v/v). Plasma samples were collected at time points (n=3 per time point) from 0-4 hours post dose, to measure concentrations of drug in plasma. Plasma samples were mixed with acetonitrile containing internal standard. The samples were vortexed, then centrifuged at 4 degrees Celsius for 15 minutes. The supernatant was diluted with water (1:2 V/V) and analyzed by LC/MS/MS for quantitative analysis. Concentration of compound in plasma was calculated for each time point.

TABLE 4

| Parameter | Ratio of 018:001 | ratio of 019:001 |
|---|---|---|
| Plasma t_{1/2} | 3.4:1 | 1.8:1 |
| Plasma Cₘₐₓ | 9.0:1 | 2.0:1 |
| Plasma AUCₗₐₛₜ | 4.2:1 | 2.2:1 |

Data presented in Table 4 as ratio of the values obtained from mesembranol(018):mesembrine(001) (“Ratio of 018:001”) or (−)-6-epimesembranol(019): (−) mesembrine(001) (“Ratio of 019:001”). A ratio >1.0 indicates an increase in t_{1/2}, Cₘₐₓ, or area under curve (AUC) in plasma compared to Compound 001 (mesembrine). Both Compound 018 and Compound 019 demonstrate significant improvements in plasma half-life (t_{1/2}), max concentration (Cₘₐₓ), as well as area under curve (AUC) compared to Compound 001. These results demonstrate improved pharmacokinetic profile of Compound 018 and Compound 019 (compared to Compound 001) leading to higher plasma levels over a longer period of time. For example, Compound 018 unexpectedly demonstrated about a 3-fold longer plasma half-life, about a 9-fold higher Cmax and about a 4-fold greater AUC(last) compared to mesembrine (Compound 001). In addition, Compound 019 unexpectedly demonstrated about a 2-fold longer plasma half-life, a 2-fold higher Cmax and about a 2-fold greater AUC(last) compared to mesembrine (Compound 001).

### Example 6 HPLC Method for Detecting Mesembrenol or Mesembrenone

Gericke et al. (Journal of Ethnopharmacology, 2022, 284, 114550) performed alkaloid profiling and quantification on an ethanolic extract of Zembrin® using a validated ultra-performance liquid chromatography-mass spectrometry coupled to a photodiode array detector (UPLC-MS-PDA), as described previously (Zhao et al., Phytochemistry, 2018, 152, 191-203). The sample comprised, in descending order, mesembrenone (47.9%) mesembrenol (32%), mesembrine (13.3%) and mesembranol (6.8%), which contributed to 3.84 μg/mg of total plant material. Using 25 mg zembrin (0.4% w/w total alkaloid) as the extract model would equate to an administered dose of mesembranol of about 7 micrograms. That would be <200 nanograms per kg in a human, which is several orders of magnitude under what would exceed the IC₅₀ of SERT.

### Example 7: Pharmacokinetic Profile of Compounds 018 and 019 in Dogs

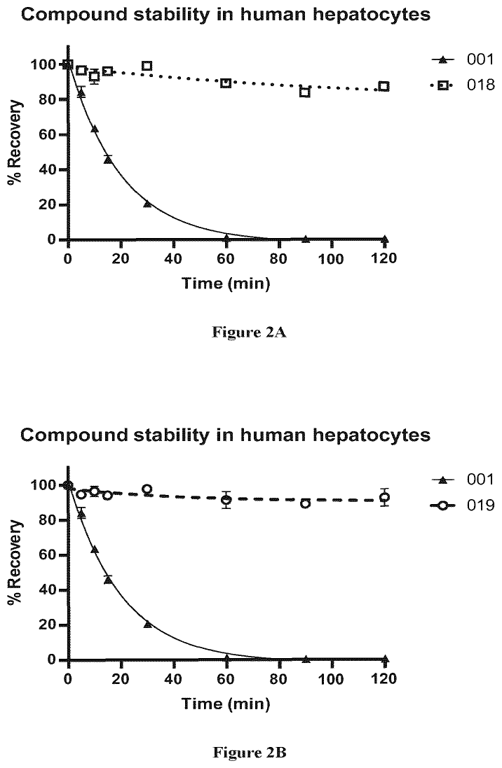

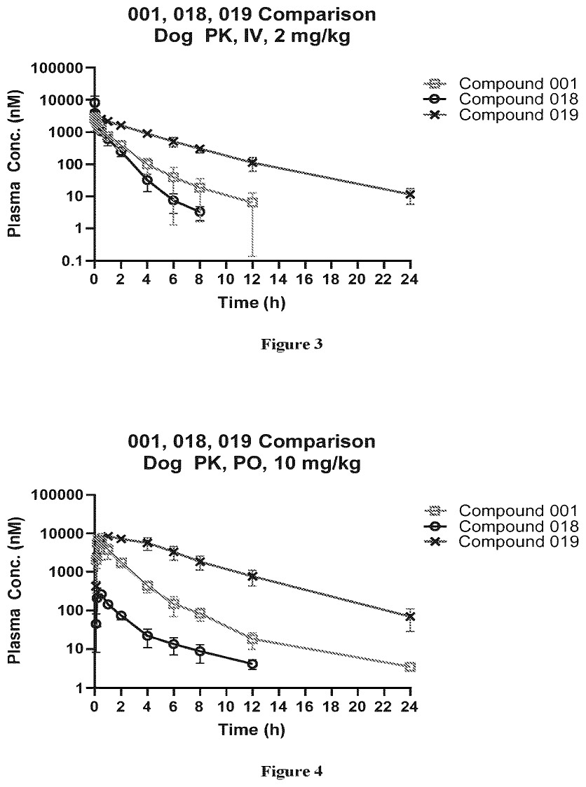

The pharmacokinetic profile of Compound 018 and Compound 019 evaluated in dogs. FIG. 3 compares the plasma concentrations (nM) of Compound 018, Compound 019 and compound 001 following IV administration of compound at 2 mg/kg FIG. 4 compares the plasma concentrations (1 M) of Compound 018, Compound 019, and Compound 001 following PO administration of compounds at 10 mg/kg.

HPLC Instrument: Shimsadzu (DGU-20A5R, Serial No: L20705621435 IX; LC-30AD Serial No: L20555611905 AE and L20555611816 AE; SIL-L30AC, Serial No: L20565605499 AE; Rack Changer II Serial No. L20585601125 SS; CTO20A: Serial No. L20205620413 CD; CBM-20A: Serial No. L20235636200 CD)
MS: AB API 5500 LC/MS/MS instrument (Serial No. EF221221812)
Column: Phenomenex Synergi 2.5 μm Polar-RP 100A. (50×3 mm)
Mobile Phase: (A) 5% Acetonitrile in Water (0.1% Formic acid); (B) 95% Acetonitrile in Water (0.1% Formic acid)
Samples: 50 μL of plasma sample+5 μL of blank solution+200 μL of acetonitrile for PPE (protein precipitation extraction).

Compounds 018 and 019:
Injection volume: 2 microliters
Flow rate: 0.6 mL/min

| Time (min) | A (%) | B (%) |
|---|---|---|
| 0.01 | 95.0 | 5.00 |
| 0.20 | 95.0 | 5.00 |
| 1.90 | 10.0 | 90.0 |
| 2.20 | 10.0 | 90.0 |
| 2.21 | 95.0 | 5.00 |
| 2.50 | 95.0 | 5.00 |

Compound 001
Injection Volume: 4 microliters

| Time (min) | A (%) | B (%) |
|---|---|---|
| 0.01 | 100 | 0.00 |
| 0.40 | 100 | 0.00 |
| 1.70 | 0.00 | 100 |
| 2.00 | 0.00 | 100 |
| 2.01 | 100 | 0.00 |
| 2.20 | 100 | 0.00 |

### Sample Preparation

The desired serial concentrations of working solutions were achieved by diluting stock solution of analyte with 50% acetonitrile in water solution. 5 μL of working solutions (5, 10, 20, 50, 100, 500, 1000, 5000, 10000 ng/mL) were added to 50 μL of the blank Beagle Dog plasma to achieve calibration standards of 0.5˜1000 ng/mL (0.5, 1, 2, 5, 10, 50, 100, 500, 1000 ng/mL) in a total volume of 55 μL. Five quality control samples at 1 ng/mL, 2 ng/mL, 5 ng/mL, 50 ng/mL and 800 ng/mL for plasma were prepared independently of those used for the calibration curves. These QC samples were prepared on the day of analysis in the same way as calibration standards.

55 μL standards, 55 μL QC samples and 55 μL unknown samples 50 μL plasma with 5 μL blank solution) were added to 200 μL of acetonitrile containing IS mixture for precipitating protein respectively. Then the samples were vortexed for 30 s. After centrifugation at 4 degree Celsius, 3900 rpm for 15 min, the supernatant was diluted 3 times with water. 2 μL for SNTX-004, SNTX-005 and 4 uL for SNTX-001 of diluted supernatant was injected into the LC/MS/MS system for quantitative analysis.

Compound information

| Compound ID | Compound 018 |
|---|---|
| MW (Free form) | 291.39 |
| FW (Salt form) | 291.39 |

Formulation Preparation
Preparation of Compound 018 PO(2 mg/kg, 5 mL/kg) Dosing
0.4 mg/mL solution of “1% NMP and 99% v/v Normal saline”
Dissolved 71.21 mg of SNTX-004 in 1.780 mL of NMP with
vortexing and sonication, then added 176.245 mL of Saline
with vortexing and sonication to obtain a solution.

Dog PK data (IV) from compounds 018, 019
and 001 (data plotted in FIG. 3)

| IV Dose | | Compound | Compound | Compound |
|---|---|---|---|---|
| 2 mg/kg | | 001 | 018 | 019 |
| PK parameters | Unit | Mean (n = 3) | Mean (n = 3) | Mean (n = 3) |
| Cl_obs | mL/min/kg | 44.07 | 47.6 | 10.82 |
| T_{1/2} | h | 1.65 | 1.78 | 3.38 |
| C₀ | ng/ml | 1226 | 6248 | 1534 |
| AUCₗₐₛₜ | h*ng/ml | 805 | 710 | 3121 |
| AUC_{Inf} | h*ng/ml | 809 | 712 | 3138 |
| AUC_% Extrap_obs | % | 0.53 | 0.26 | 0.52 |
| MRT_{Inf}_obs | h | 1.41 | 0.71 | 3.86 |
| AUCₗₐₛₜ/D | h*mg/mL | 402 | 355 | 1560 |

Dog PK data (PO) from compounds 018, 019, 001
(data plotted in FIG. 4)

| PO Dose | | Compound | Compound | Compound |
|---|---|---|---|---|
| 10 mg/kg | | 001 | 018 | 019 |
| PK parameters | Unit | Mean (n = 3) | Mean (n = 3) | Mean (n = 3) |
| T_{1/2} | h | 2.99 | 5.95 | 3.33 |
| Tₘₐₓ | h | 0.28 | 0.28 | 0.83 |
| Cₘₐₓ | ng/ml | 1878.0 | 84.4 | 2553.3 |
| AUCₗₐₛₜ | h*ng/ml | 3106.9 | 144.0 | 14732.2 |
| AUC_{Inf} | h*ng/ml | 3113.0 | 151.4 | 14833.9 |
| AUC_% Extrap_obs | % | 0.25 | 5.10 | 0.64 |
| MRT_{Inf}_obs | h | 1.81 | 3.95 | 4.85 |
| AUCₗₐₛₜ/D | h*mg/mL | 310.7 | 14.4 | 1473.2 |
| F | % | 76.9 | 4.3 | 94.6 |

Compound 019 showed excellent oral bioavailability. Poor oral bioavailability of Compound 018 was predicted based on in vitro clearance data in dog hepatocytes.

### Example 8: Antidepressant Sig-Nature of Compound 018 and Compound 019 in Mice Using Smartcube System

To confirm that compound 018 and Compound 019 has antidepressant effects, the SmartCube® system (Psychogenics, Inc., Paramus, NJ) was used. This system uses features derived from mouse behavioral data to classify compounds for their ability to treat neuro-psychiatric symptoms by comparing the features to a proprietary reference database of behavioral feature sets that are linked to classes of marketed drugs known to treat neuro-psychiatric symptoms. Thus the system can be used as a model to identify the psychiatric effect of a compound by comparing the effects of the compounds against drugs with known validated effects. By comparing the responses of animals to known drugs, the test drug can be categorized according to its function; for example, hallucinogen, anxiogenic, analgesic, cognitive enhancer, psychostimulant, mood stabilizer, high dose anti-psychotic, anti-psychotic, sedative/hypnotic, anxiolytic, high dose antidepressant, antidepressant.

Once all features are extracted from the raw data through an automated pipeline, proprietary bioinformatics algorithms are used to decorrelate groups of features and find the combination of values that best separate different groups of interest. For each compound, at each dose, they system provides a probability that the drug is active and breaks down such putative activity into the different classes of interest.

The SmartCube® system is designed to and can successfully measure numerous spontaneous behaviors and response to challenges in the same testing environment.

The hardware includes force sensors and a number of aversive stimuli to elicit behavior. Three high-resolution video cameras provide constant 3D view of the mouse in the SmartCube® apparatus (SC) throughout the entire testing period.

Mice were tested in the SmartCube® system where they were exposed to a sequence of challenges during the 45-minute test period. The cubes were cleaned between each run.

For class and subclass analysis, a reference data set has been built from hundreds of drug doses in multiple drug classes plus a control group. Each reference drug was tested at multiple doses appropriate for that drug in mice. The best performing classifiers were chosen from our evaluation tests and two separate types of classifiers were built that make independent predictions at drug class and subclass levels. The Class consists of drugs that are currently in the market or have been clinically validated for that specific indication. The sub-class consists of both marketed drugs and other compounds that have been mechanistically validated and is a larger set than the Class.

Data was processed using proprietary computer vision and data mining algorithms and the results were compared to signatures of the reference compounds in our database. Multiple analyses of the data was performed to quantitatively produce independent predictions of drug class, and drug subclass. The behavioral signatures of the test drug were evaluated using these classifiers to predict potential therapeutic utility.

A variety of analytical tools are available for follow up analysis to answer different questions. For instance, a differentiation between typical and atypical antipsychotics, or similarity to mood stabilizers may be of interest to widen the therapeutic indications for a lead compound. Whereas in the class analysis used to screen compounds a dozen pharmacological classes were assessed at a time (class analysis), in the cloud analysis just 2-4 classes of interest can be focused on. For example, a classifier can be trained using supervised training to separate typical from atypical antipsychotics, and then use a novel drug as a test set and classify the corresponding samples in the space created by the training. In this way, not only can similarity to one or the other classes be quantified, but also visualize it. As described above, the outcome of a SmartCube® run is a set of more than 2000 features. By creating independent combinations of the original features, it is possible to reduce data complexity and extract the derived features that maximally separate groups of interest. Similar to principal component analysis, the most important composite axes that best separate the groups under analysis can be found. To create a useful visualization tool, Gaussian distributions representing the groups of interest in the 2D space created by these major axes can be plotted, where the width is the standard deviation given by the mouse-to-mouse variability. The separation between the groups or classes is a measure of similarity or lack thereof (discrimination), and its statistical significance can be calculated by estimating the probability that the result is due to chance.

Compound 018 was behaviorally inactive (<40%) at 3 mg/kg. At higher doses (10, 30 and 100 mg/kg) the compound showed a robust antidepressant-like signature with a small portion showing psychostimulant-like signature in both class and subclass. Subclass analysis showed that antidepressant signature was similar to that of an SSRI.

Compound 019 showed low behaviorally activity at 3 mg/kg. At higher doses (10, 30 and 100 mg/kg) the compound showed a mixed antidepressant and psychostimulant-like signatures in both class and subclass. The antidepressant signature was similar to that of an SSRI at all doses and at 100 mg/kg it was a mix of SSRI and SNRI.

The invention claimed is:

1-20. (canceled)

21. A method of treating anxiety or depression, the method comprising administering to a patient in need thereof a therapeutically effective amount of a pharmaceutical composition comprising (−) 6-epi-mesembranol having an inhibition potency (IC₅₀) at the 5-HT serotonin transporter (SERT) of less than about 30 nM (as measured by the SERT Inhibition Assay of Example 3A) without inhibiting phosphodiesterase-4 (PDE4) subtypes PDE4 A1A, PDE4 B2, PDE4 C1 or PDE4 D2 by more than 5% at a concentration of 10 micromolar (as measured by the PDE4 Inhibition Assay of Example 3B).

22. The method of claim 21, wherein the pharmaceutical composition has an inhibition potency (IC₅₀) at the 5-HT serotonin transporter (SERT) of less than about 15 nM (as measured by the SERT Inhibition Assay of Example 3A).

23. The method of claim 21, wherein the pharmaceutical composition has a ratio of PDE4/SERT inhibition values of at least 100:1.

24. The method of claim 21, wherein the pharmaceutical composition has a ratio of PDE4/SERT inhibition values of at least 500:1.

25. The method of claim 21, wherein the pharmaceutical composition contains no more than about 1% of an alkaloid selected from (−) mesembrine, mesembrenone, mesembrenol, or a combination thereof.

26. The method of claim 21, wherein at least 90% of the total alkaloid content in the pharmaceutical composition is 6-epi-mesembranol.

27. The method of claim 21, wherein the pharmaceutical composition contains less than about 70 micrograms of mesembrenone and mesembrenol per 11.3 mg total alkaloid content; and the pharmaceutical composition contains less than about 23 micrograms of (−) mesembrine per 11.3 mg total alkaloid content.

28. The method of claim 21, wherein the pharmaceutical composition comprises less than about 0.5% of mesembrine or mesembrenone.

29. The method of claim 21, wherein the pharmaceutical composition is administered orally.

30. The method of claim 21, wherein (a) at least 90% of the total alkaloid content in the pharmaceutical composition is (−) 6-epi-mesembranol; and (b) the pharmaceutical composition contains no more than about 1% of an alkaloid selected from (−) mesembrine, mesembrenone, mesembrenol, or a combination thereof.

31. The method of claim 30, wherein (a) the pharmaceutical composition contains less than about 70 micrograms of mesembrenone and mesembrenol per 11.3 mg total alkaloid content; and the pharmaceutical composition contains less than about 23 micrograms of (−) mesembrine per 11.3 mg total alkaloid content; and (b) the pharmaceutical composition comprises less than about 0.5% of mesembrine or mesembrenone.

32. The method of claim 31, wherein the pharmaceutical composition has a ratio of PDE4/SERT inhibition values of at least 100:1.

33. The method of claim 21, wherein the patient is diagnosed with Social Anxiety Disorder (SAD).

34. The method of claim 21, wherein the patient is diagnosed with Generalized Anxiety Disorder (GAD).

35. The method of claim 21, wherein the patient is diagnosed with depression.

36. A method of treating anxiety or depression, the method comprising administering to a patient in need thereof a therapeutically effective amount of a pharmaceutical composition comprising Compound (019): 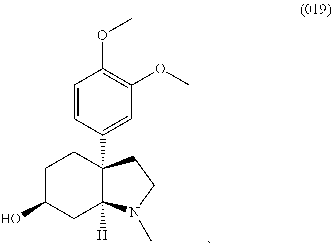 [SMILES: COc1ccc([C@@]23CC[C@H](O)C[C@@H]2N(C)CC3)cc1OC]
or a pharmaceutically acceptable salt thereof, wherein at least 90% of the total alkaloid content in the pharmaceutical composition is Compound (019), or a pharmaceutically acceptable salt thereof, and the pharmaceutical composition contains no more than about 1% of any alkaloid selected from (−) mesembrine, mesembrenone, mesembrenol, or any combination thereof.

37. The method of claim 36, wherein the pharmaceutical composition contains (a) less than about 70 micrograms of mesembrenone and mesembrenol per 11.3 mg total alkaloid content; and the pharmaceutical composition contains less than about 23 micrograms of (−) mesembrine per 11.3 mg total alkaloid content; and (b) less than about 0.5% of mesembrine or mesembrenone.

38. The method of claim 37, wherein the pharmaceutical composition has an inhibition potency (IC₅₀) at the 5-HT serotonin transporter (SERT) of less than about 30 nM (as measured by the SERT Inhibition Assay of Example 3A) without inhibiting phosphodiesterase-4 (PDE4) subtypes PDE4 A1A, PDE4 B2, PDE4 C1 or PDE4 D2 by more than 5% at a concentration of 10 micromolar (as measured by the PDE4 Inhibition Assay of Example 3B).

39. The method of claim 38, wherein the patient is diagnosed with Social Anxiety Disorder (SAD), Generalized Anxiety Disorder (GAD) or depression, and the pharmaceutical composition is orally administered to the patient in need thereof.

40. An anxiolytic pharmaceutical composition, comprising Compound (019): 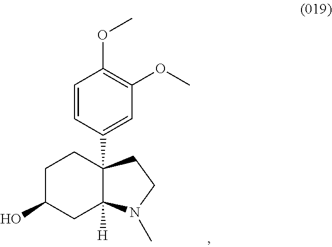 [SMILES: COc1ccc([C@@]23CC[C@H](O)C[C@@H]2N(C)CC3)cc1OC]
or a pharmaceutically acceptable salt thereof, and a pharmaceutically acceptable excipient; wherein
the pharmaceutical composition contains no more than about 3% of an alkaloid selected from (−) mesembrine, mesembrenone, mesembrenol, or any combination thereof;
Compound (019) is at least 90% of the total alkaloid content in the pharmaceutical composition; and
the ratio of phosphodiesterase-4 (PDE4) to 5-HT serotonin transporter (SERT) inhibition values of the pharmaceutical composition (as measured by the SERT Inhibition Assay of Example 3A and the PDE4 Inhibition Assay of Example 3B) is at least 10:1 and the pharmaceutical composition has a % inhibition (as measured by the PDE4 Inhibition Assay of Example 3B) of less than about 6% for at least one of PDE4 A1A, PDE4 B2, PDE4 C1, and PDE4 D2.
